(19) United States
(12) Patent Application Publication
(10) Pub. No.: US 2021/0298615 A1
(43) Pub. Date: Sep. 30, 2021

(54) BIOLOGICAL INFORMATION ACQUISITION DEVICE, HEAD-MOUNTED INFORMATION PRESENTATION DEVICE, AND BIOLOGICAL INFORMATION ACQUISITION METHOD

(71) Applicant: Sony Corporation, Tokyo (JP)

(72) Inventors: Yukinojo KITAKAMI, Tokyo (JP); Atsushi ITO, Tokyo (JP); Takanori ISHIKAWA, Tokyo (JP)

(21) Appl. No.: 17/260,699

(22) PCT Filed: Jul. 18, 2019
(86) PCT No.: PCT/JP2019/028184

§ 371 Date: Jan. 15, 2021

(30) Foreign Application Priority Data
Aug. 9, 2018 (JP) 2018-150797

(51) Int. Cl. A61B 5/0285; A61B 5/00; A61B 5/11

(52) U.S. Cl. CPC A61B 5/0285; A61B 5/6803; A61B 5/7405; A61B 5/742; A61B 5/1116

(57) ABSTRACT

An object is to provide a technique for removing fluctuations caused by a change in posture of a target from which a blood flow velocity is acquired when measuring the blood flow velocity.

The present technology provides a biological information acquisition device including: a blood flow velocity measurement unit that is installed on a head; a detection unit that detects a position change amount of the blood flow velocity measurement unit, a moving velocity of the blood flow velocity measurement unit, or the position change amount and the moving velocity of the blood flow velocity measurement unit; a correction information generation unit that generates correction information of a blood flow velocity on the basis of the position change amount, the moving velocity, or the position change amount and the moving velocity; and a correction unit that corrects the blood flow velocity measured by the blood flow velocity measurement unit using the correction information. Furthermore, the present technology also provides a head-mounted information presentation device and a biological information acquisition method.

### TECHNICAL FIELD

The present technology relates to a biological information acquisition device, a head-mounted information presentation device, and a biological information acquisition method. More specifically, the present technology relates to a biological information acquisition device, a head-mounted information presentation device, and a biological information acquisition method that correct a measured blood flow velocity.

### BACKGROUND ART

Biological information such as blood flow, blood pressure, heart rate, and body temperature is used to grasp the health state and/or psychological state of a person. Various devices have been proposed so far in order to acquire the biological information. For example, as a method for measuring blood flow, a method using laser Doppler flowmetry (hereinafter, also referred to as LDF), a method using ultrasonic waves, and a method using electromagnetic induction can be mentioned.

In order to acquire more accurate biological information, the measured biological information can be corrected. As an example of the technique related to such correction, for example, Patent Document 1 below discloses a blood pressure correction information generation device including a specific wrist position change period detection unit and a specific blood pressure correction information generation unit. The blood pressure correction information generated by the device corrects a measurement error of the blood pressure information caused by a change in the position of the wrist in the wrist-mounted blood pressure measurement device.

### CITATION LIST

### Patent Document

Patent Document 1: Japanese Patent Application Laid-Open No. 2017-121273

### SUMMARY OF THE INVENTION

### Problems to be Solved by the Invention

The biological information not only fluctuates depending on the health state and/or the psychological state, but also fluctuates depending on the posture or moving velocity of a target from which the biological information is acquired. Therefore, in order to more accurately grasp the health state and/or the psychological state on the basis of the biological information, it is desirable that the biological information be corrected to remove fluctuations caused by a change in the posture of a biological information acquisition target or the moving velocity of the biological information acquisition target.

An object of the present technology is to provide a technique for removing fluctuations caused by a change in the posture of a target or the moving velocity of the target from which the blood flow velocity is acquired when measuring the blood flow velocity.

### Solutions to Problems

The present technology provides a biological information acquisition device including:

a blood flow velocity measurement unit that is installed on a head;

a detection unit that detects a position change amount of the blood flow velocity measurement unit, a moving velocity of the blood flow velocity measurement unit, or the position change amount and the moving velocity of the blood flow velocity measurement unit;

a correction information generation unit that generates correction information of a blood flow velocity on the basis of the position change amount, the moving velocity, or the position change amount and the moving velocity; and

a correction unit that corrects the blood flow velocity measured by the blood flow velocity measurement unit using the correction information.

According to one embodiment of the present technology, the position change amount may be the amount of change in the inclination of the blood flow velocity measurement unit or the amount of change in the height of the blood flow velocity measurement unit. According to one embodiment of the present technology, the position change amount can include an amount of change in inclination of the blood flow velocity measurement unit, and the inclination can include an angle projected onto a plane passing the blood flow velocity measurement unit and a center line of the head.

According to one embodiment of the present technology, the moving velocity can include an angular velocity of the blood flow velocity measurement unit or a velocity of the blood flow velocity measurement unit.

Particularly preferably, the moving velocity can be the angular velocity of the blood flow velocity measurement unit.

According to one embodiment of the present technology, the correction information generation unit can generate

correction information for canceling a fluctuation in blood flow velocity caused by a change in position of the head,

correction information for canceling a fluctuation in blood flow velocity caused by the moving velocity of the head, or

correction information obtained by adding up the two pieces of correction information.

According to one embodiment of the present technology, the correction information generation unit can generate

correction information on the basis of the position change amount, a position characteristic correction parameter set on the basis of a relationship between a position of the blood flow velocity measurement unit and a blood flow velocity fluctuation amount, and/or a transient characteristic correction parameter related to transient characteristics of the blood flow velocity, and/or generate

correction information on the basis of the moving velocity, a velocity characteristic correction parameter set on the basis of a relationship between the moving velocity of the blood flow velocity measurement unit and the blood flow velocity fluctuation amount, and/or the transient characteristic correction parameter related to the transient characteristics of the blood flow velocity.

According to one embodiment of the present technology, the biological information acquisition device may further include a position characteristic correction parameter setting unit that sets and/or updates the position characteristic correction parameter.

The position characteristic correction parameter setting unit can set and/or update the position characteristic correction parameter before performing correction by the correction unit.

The position characteristic correction parameter setting unit can set and/or update the position characteristic correction parameter on the basis of the relationship between the position change amount in a case where the position of the blood flow velocity measurement unit is changed to various different positions from the reference position and the blood flow velocity measured at each of the various different positions.

According to one embodiment of the present technology, the biological information acquisition device may further include a velocity characteristic correction parameter setting unit that sets and/or updates the velocity characteristic correction parameter.

The velocity characteristic correction parameter setting unit can set and/or update the velocity characteristic correction parameter before performing correction by the correction unit.

The velocity characteristic correction parameter setting unit can set and/or update the velocity characteristic correction parameter on the basis of a relationship between various moving velocities of the blood flow velocity measurement unit and a blood flow velocity measured at each of the various moving velocities.

According to one embodiment of the present technology, the biological information acquisition device may further include a transient characteristic correction parameter setting unit that sets and/or updates the transient characteristic correction parameter.

The transient characteristic correction parameter setting unit can set and/or update the transient characteristic correction parameter before performing correction by the correction unit.

The transient characteristic correction parameter setting unit can set and/or update the transient characteristic correction parameter by fitting a blood flow velocity estimated value estimated by using the position characteristic correction parameter to temporal blood flow velocity data temporally measured in a process of changing the position of the blood flow velocity measurement unit to another position from a reference position.

According to one embodiment of the present technology, the correction unit can use the correction information to cancel a fluctuation in blood flow velocity caused by a change in position of the head and/or a fluctuation in blood flow velocity caused by a moving velocity of the head.

Furthermore, the present technology also provides a head-mounted information presentation device including:

a blood flow velocity measurement unit that is installed on a head;

a detection unit that detects a position change amount of the blood flow velocity measurement unit, a moving velocity of the blood flow velocity measurement unit, or the position change amount and the moving velocity of the blood flow velocity measurement unit;

a correction information generation unit that generates correction information on the basis of the position change amount, the moving velocity, or the change amount and the moving velocity; and

a correction unit that corrects the blood flow velocity measured by the blood flow velocity measurement unit using the correction information.

Furthermore, the present technology also provides a biological information acquisition method including:

a blood flow velocity measurement process of measuring the blood flow velocity of a head by a blood flow velocity measurement device installed on the head,

a detection process of detecting a position change amount of the blood flow velocity measurement device, a moving velocity of the blood flow velocity measurement device, or the position change amount and the moving velocity of the blood flow velocity measurement device,

a correction information generation process of generating correction information on the basis of the position change amount, the moving velocity, or the change amount and the moving velocity, and

a correction process of correcting the blood flow velocity measured by the blood flow velocity measurement device using the correction information.

According to the present technology, the measured blood flow velocity can be corrected so as to remove the fluctuation caused by a change in the posture of a measurement target or the moving velocity of the measurement target.

Note that effects provided by the present technology are not necessarily limited to effects described herein, but may also be any of those described in the present description.

### BRIEF DESCRIPTION OF DRAWINGS

FIG. 1 is an example of a block diagram of a biological information acquisition device according to the present technology.

FIG. 2 is a model diagram for explaining a change in blood flow velocity.

FIG. 3 is a diagram showing an example of a position where a blood flow velocity measurement device is installed.

FIG. 4 is a diagram for explaining a change in inclination of a head.

FIG. 5 is a model diagram for explaining resistance and gravity acting on a red blood cell.

FIG. 6 is a diagram for explaining an example of a method of setting a position characteristic correction parameter.

FIG. 7 is a model diagram for explaining a change in blood flow velocity.

FIG. 8 is a diagram showing an example of a configuration of a head-mounted information presentation device according to the present technology.

FIG. 9 is a diagram showing an example of a configuration of a head-mounted information presentation device according to the present technology.

FIG. 10 is an example of a flow chart of biological information acquisition processing according to the present technology.

FIG. 11 is a graph showing a relationship between blood flow velocity and head inclination.

FIG. 12 is a graph showing blood flow velocity before and after correction.

FIG. 13 is an example of a block diagram of a biological information acquisition device according to the present technology.

FIG. 14 is a model diagram for explaining resistance and gravity acting on a red blood cell.

FIG. 15 is a diagram for explaining an example of a method of setting a velocity characteristic correction parameter.

FIG. 16 is an example of a block diagram of a biological information acquisition device according to the present technology.

FIG. 17 is an example of a flow chart of biological information acquisition processing according to the technology.

FIG. 18 is an example of a flow chart of biological information acquisition processing according to the technology.

FIG. 19 is a graph showing blood flow velocity before and after correction.

### MODE FOR CARRYING OUT THE INVENTION

Preferred aspects for carrying out the present technology are described below. Note that embodiments described below indicate representative embodiments of the present technology, and the scope of the present technology is not limited to such embodiments. Note that the present technology will be described in the following order.
1. First embodiment (biological information acquisition device)

(1) Description of the first embodiment

(2) Example of configuration of the biological information acquisition device (correction based on position change amount of head)

(2-1) Correction information based on the amount of change in inclination

(2-2) Correction information based on the amount of change in height

(3) Example of configuration of the biological information acquisition device (correction based on moving velocity of head)

(3-1) Correction information based on angular velocity

(4) Example of configuration of the biological information acquisition device (correction based on position change amount and moving velocity of head)
2. Second embodiment (head-mounted information presentation device)

(1) Description of the second embodiment

(2) Configuration of the head-mounted information presentation device
3. Third embodiment (biological information acquisition method)

(1) Description of the third embodiment

(2) Example of the third embodiment (biological information acquisition method using position change amount of head)

(3) Example of the third embodiment (biological information acquisition method using moving velocity of head)

(4) Example of the third embodiment (biological information acquisition method using position change amount and moving velocity of head)
4. Example

(1) Correction based on position change amount

(2) Correction based on position change amount and moving velocity

### 1. FIRST EMBODIMENT (BIOLOGICAL INFORMATION ACQUISITION DEVICE)

(1) Description of the First Embodiment

A biological information acquisition device according to the present technology generates correction information on the basis of the position change amount of a blood flow velocity measurement unit installed on a head, the moving velocity of the blood flow velocity measurement unit, or both the position change amount and the moving velocity, and then corrects the blood flow velocity measured by the blood flow velocity measurement unit by using the correction information. Therefore, from the measured blood flow velocity of the head, the fluctuations in the blood flow velocity caused by a change in the position of the head and/or the moving velocity of the head, i.e., fluctuations in the blood flow velocity caused by a change in the posture of the person and/or the transition degree of the person can be removed. Thus, the biological information acquisition device according to the present technology can obtain blood flow velocity data that enables a more accurate grasp the health state and/or the psychological state of a person.

(2) Example of Configuration of the Biological Information Acquisition Device (Correction Based on Position Change Amount)

According to one embodiment of the present technology, the biological information acquisition device generates correction information on the basis of the position change amount of a blood flow velocity measurement unit installed on a head, and then corrects the blood flow velocity measured by the blood flow velocity measurement unit by using the correction information.

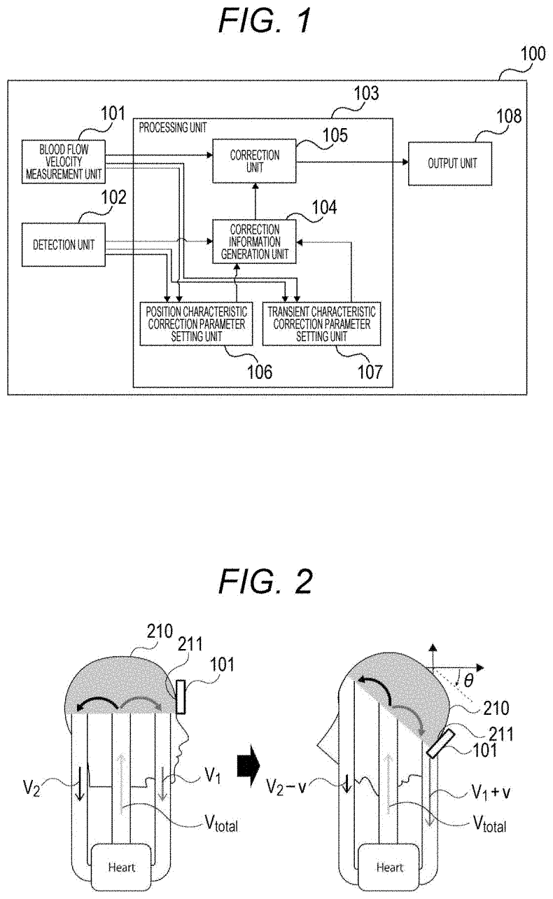

An example of the biological information acquisition device of this embodiment will be described below with reference to FIG. 1. FIG. 1 is an example of a block diagram of the biological information acquisition device of this embodiment.

As shown in FIG. 1, a biological information acquisition device 100 includes a blood flow velocity measurement unit 101, a detection unit 102, a processing unit 103, and an output unit 108. The processing unit 103 includes a correction information generation unit 104, a correction unit 105, a position characteristic correction parameter setting unit 106, and a transient characteristic correction parameter setting unit 107.

The blood flow velocity measurement unit 101 is installed on the head. The blood flow velocity measurement unit 101 may be configured to be able to measure the blood flow velocity of the head. A blood flow velocity measuring method used by the blood flow velocity measurement unit 101 may be a method known in the art, for example, any of a laser Doppler flowmetry (LDF) method, an ultrasonic method, and an electromagnetic induction utilization method. The blood flow velocity measurement unit 101 can include a blood flow velocity measurement device that measures the blood flow velocity by any of these methods. The blood flow velocity measurement device may be installed at any position on the head, for example, on the frontal head portion, the side head portion, or the rear head portion.

A more specific example of the blood flow velocity measurement device for measuring the blood flow velocity by the LDF method will be described below. The blood flow velocity device can include, for example, a laser light irradiation unit that irradiates the skin of the head with laser light, a detection unit that detects the light generated by the irradiation of the skin with the laser light, and a blood flow velocity calculation unit that calculates the blood flow velocity on the basis of the wavelength of the scattered light detected by the detection unit. Within the scattered light generated by the irradiation of the laser light, the frequency of the scattered light scattered by a stationary biological tissue is the same as the frequency of the irradiated laser light, while the frequency of scattered light scattered by scattering substances (mainly red blood cells) that move in the blood vessels of the skin of the head is subject to a slight Doppler shift. The interference light generated by the interference of these two scattered light is detected by the detection unit. The blood flow velocity calculation unit calculates the blood flow velocity by performing frequency analysis processing on the optical beat of the interference light.

The detection unit 102 detects the position change amount of the blood flow velocity measurement unit 101, particularly the position change amount of the blood flow velocity measurement device. In the present technology, the amount of change can be the amount of change in the inclination of the blood flow velocity measurement unit or the amount of change in the height of the blood flow velocity measurement unit.

The detection unit 102 can include, for example, an acceleration sensor, a gyro sensor, or a barometric pressure sensor. These sensors used in the present technology may be those known in the art. For example, any of these sensors can be provided in the biological information acquisition device 100 so that the position change is the same as the position change of the blood flow velocity measurement unit 101. For example, the positional relationship between the blood flow velocity measurement unit 101 and the detection unit 102 can be fixed. Therefore, the position change amount detected by the detection unit 102 can be treated as the position change amount of the blood flow velocity measurement unit 101.

Alternatively, the detection unit 102 may be a combination of a marker indicating the position of the blood flow velocity measurement unit 101 and a marker recognition device that recognizes the marker. The marker may be attached to the blood flow velocity measurement unit 101, or may be attached to the biological information acquisition device 100 so that the position change is the same as the position change of the blood flow velocity measurement unit 101. The marker recognition device can include an imaging element. By processing a still image or a moving image obtained by the imaging element, the position change amount of the marker can be measured. The position change amount of the marker can be treated as the position change amount of the blood flow velocity measurement unit 101.

The processing unit 103 can include a processor such as a CPU and a memory such as RAM and/or ROM. A program for causing the device to execute the biological information acquisition method according to the present technology, a position characteristic correction parameter and a transient characteristic correction parameter described below, a program for setting or updating these parameters, and the like can be stored in the memory. The function of the processing unit 103 can be realized by the processor.

The correction information generation unit 104 generates correction information on the basis of the position change amount of the blood flow velocity measurement unit 101 detected by the detection unit 102. Since the correction information is generated on the basis of the position change amount of the blood flow velocity measurement unit 101, it is suitable for canceling a change (increase/decrease) in the blood flow velocity caused by a change in the position of the head.

That is, according to one embodiment of the present technology, the correction information generation unit 104 can generate correction information for canceling fluctuations (for example, increase or decrease) of the blood flow velocity caused by a change in the position of the head. For example, the correction information generation unit 104 can generate correction information for canceling an increase in the blood flow velocity caused by lowering in the position of the head or correction information for canceling a decrease in the blood flow velocity caused by raising in the position of the head. More specifically, the correction information generation unit 104 can generate correction information on the basis of the position change amount, the position characteristic correction parameter set on the basis of the relationship between the position of the blood flow velocity measurement unit and the blood flow velocity fluctuation amount and/or the transient characteristic correction parameter related to transient characteristics of the blood flow velocity. More specific examples of the generated correction information will be described in (2-1) and (2-2) below.

The correction unit 105 corrects the blood flow velocity measured by the blood flow velocity measurement unit 101 by using the correction information generated by the correction information generation unit 104.

For example, in a case where the blood flow velocity has increased due to lowering in the position of the head, the correction unit 105 subtracts the increase from the measured blood flow velocity by using the correction information generated by the correction information generation unit 104. Alternatively, in a case where the blood flow velocity has decreased due to raising in the position of the head, the correction unit 105 adds the decrease to the measured blood flow velocity by using the correction information generated by the correction information generation unit 104. As described above, in the present technology, the correction unit 105 can cancel the fluctuation of the blood flow velocity caused by the change in the position of the head by using the correction information. For example, the correction unit 105 can use the correction information to cancel the increase in the blood flow velocity caused by lowering in the position of the head or cancel the decrease in the blood flow velocity caused by raising in the position of the head.

The position characteristic correction parameter setting unit 106 sets and/or updates the position characteristic correction parameter set on the basis of the relationship between the position of the blood flow velocity measurement unit and the blood flow velocity fluctuation amount.

The position characteristic correction parameter setting unit 106 can set and/or update the position characteristic correction parameter before, for example, performing correction by the correction unit. The biological information acquisition device 100 may have a predetermined position characteristic correction parameter in advance or may not have the position characteristic correction parameter in advance.

In a case where the biological information acquisition device 100 has a predetermined position characteristic correction parameter in advance, the position characteristic correction parameter setting unit 106 may update the position characteristic correction parameter before the blood flow velocity is corrected according to the present technology. In a case where the biological information acquisition device 100 has a predetermined position characteristic correction parameter in advance, the position characteristic correction parameter may not be updated. For example, the position characteristic correction parameter set at the time of shipment of the biological information acquisition device 100 may be used as it is, or the position characteristic correction parameter set when a certain user uses the biological information acquisition device 100 for the first time may be used as it is at the next use (when the biological information acquisition device or the blood flow velocity measurement device is mounted after the second time).

In a case where the biological information acquisition device 100 does not have a position characteristic correction parameter in advance, the position characteristic correction parameter setting unit 106 may set the position characteristic correction parameter before the blood flow velocity is corrected according to the present technology.

In this way, by setting or updating the position characteristic correction parameter before the blood flow velocity is corrected according to the present technology, the position characteristic correction parameter according to a target (particularly person) whose blood flow is measured by the biological information acquisition device according to the present technology can be set or updated. More accurate correction information can be generated by the set or updated position characteristic correction parameter, and then, more appropriate correction of blood flow velocity can be performed on the basis of the more accurate correction information.

While the biological information acquisition device according to the present technology is mounted on the target, the position characteristic correction parameter may be updated at predetermined time intervals or for each buffer unit. More specifically, the update may be performed, for example, every 3 minutes to 3 hours, particularly every 5 minutes to 2 hours, and more particularly every 10 minutes to 1 hour. The buffer unit refers to one sample unit of the blood flow velocity measured by the blood flow velocity measurement device. For example, the predetermined number of times of measuring the blood flow velocity can be one unit, or the predetermined time interval in which the blood flow velocity is measured can be one unit. By updating the position characteristic correction parameter at predetermined time intervals or for each buffer unit in this way, for example, in a case where the blood flow velocity is acquired by the biological information acquisition device according to the present technology for a long period of time, a more appropriate position characteristic correction parameter enables more appropriate correction of blood flow velocity.

The setting and/or update of the position characteristic correction parameter will be described in more detail below in (2-1) or (2-2).

The transient characteristic correction parameter setting unit 107 sets and/or updates the transient characteristic correction parameter relating to the transient characteristics of the blood flow velocity.

The transient characteristic correction parameter setting unit 107 can set and/or update the transient characteristic correction parameter, for example, before the blood flow velocity measurement unit performs correction by the correction unit. The biological information acquisition device 100 may have a predetermined transient characteristic correction parameter in advance or may not have the transient characteristic correction parameter in advance.

In a case where the biological information acquisition device 100 has a predetermined transient characteristic correction parameter in advance, the transient characteristic correction parameter setting unit 107 may update the transient characteristic correction parameter before the blood flow velocity is corrected according to the present technology. In a case where the biological information acquisition device 100 has a predetermined transient characteristic correction parameter in advance, the transient characteristic correction parameter may not be updated. For example, the transient characteristic correction parameter set at the time of shipment of the biological information acquisition device 100 may be used as it is, or the transient characteristic correction parameter set when a certain user uses the biological information acquisition device 100 for the first time may be used as it is at the next use (when the biological information acquisition device or the blood flow velocity measurement device is mounted after the second time).

In a case where the biological information acquisition device 100 does not have a transient characteristic correction parameter in advance, the transient characteristic correction parameter setting unit 107 may set the transient characteristic correction parameter before the blood flow velocity is corrected according to the present technology.

In this way, by setting or updating the transient characteristic correction parameter before the blood flow velocity is corrected according to the present technology, the transient characteristic correction parameter according to a target (particularly person) to which the biological information acquisition device according to the present technology is applied can be set or updated. More accurate correction information can be generated by the set or updated transient characteristic correction parameter, and then, more appropriate correction of blood flow velocity can be performed on the basis of the more accurate correction information.

While the biological information acquisition device according to the present technology is mounted on the target, the transient characteristic correction parameter may be updated at predetermined time intervals or for each buffer unit. More specifically, the update may be performed, for example, every 3 minutes to 3 hours, particularly every 5 minutes to 2 hours, and more particularly every 10 minutes to 1 hour. Therefore, for example, in a case where the blood flow velocity is acquired by the biological information acquisition device according to the present technology for a long period of time, a more appropriate transient characteristic correction parameter enables more appropriate correction of blood flow velocity.

The setting and/or update of the transient characteristic correction parameter will be described in more detail below in (2-1) or (2-2).

The output unit 108 can output the measured or corrected blood flow velocity, and output the psychological state or health state of the measurement target determined on the basis of the measured or corrected blood flow velocity, or output a video or a sound based on the measured or corrected blood flow velocity. The output unit 108 can include, for example, a printing device, an image display device, or a sound output device for outputting the blood flow velocity, the psychological state, the health state, the video, or the sound. The video or sound according to the blood flow velocity can be, for example, a video or a sound for notifying the measurement target of a case where the blood flow velocity is out of a predetermined numerical range, or a video or a sound for encouraging the measurement target to take a break.

The biological information acquisition processing by the biological information acquisition device 100 may be performed as described, for example, in 3. “(2) Example of the third embodiment (biological information acquisition method using position change amount of head)”below.

(2-1) Correction Information Based on the Amount of Change in Inclination

According to one embodiment of the present technology, the correction information generation unit 104 can generate the correction information of the blood flow velocity on the basis of the amount of change in the inclination of the blood flow velocity measurement unit. In this embodiment, the inclination can be, for example, an angle projected onto a plane passing the blood flow velocity measurement unit and the center line of the head. The generation of the correction information will be described in more detail below.

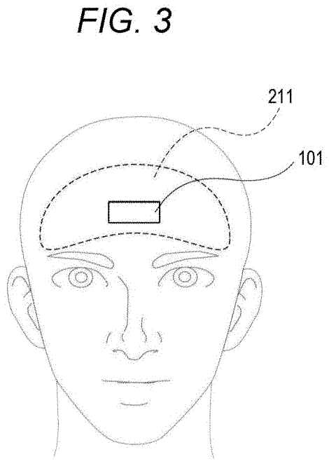

The velocity of blood flow flowing through a blood vessel at a certain position of the head changes as the position of the head changes. The change in the blood flow velocity caused by the change in the position of the head (the increase in the blood flow velocity and the decrease in the blood flow velocity) will be described below with reference to FIG. 2. FIG. 2 is a model diagram for explaining a change in the blood flow velocity.

As shown on the left side of FIG. 2, the blood flow velocity measurement unit 101 is installed on a frontal head portion (forehead) 211 of a person 210. The blood flow velocity measurement unit 101 includes, for example, an LDF type blood flow velocity measurement device. As shown in FIG. 3, the blood flow velocity measurement unit 101 is arranged at the center of the frontal head portion 211. The blood flow velocity measurement unit 101 can measure the blood flow velocity of the frontal head portion 211 by the LDF method.

On the left side of FIG. 2, the person 210 faces the front, and the position of the blood flow velocity measurement unit 101 in this state is used as a reference position. In the case shown on the left side of FIG. 2, for example, a blood flow flowing from the heart to the head is defined as Vₜₒₜₐₗ, a blood flow velocity in the skin at a certain position on the frontal head portion measured by the blood flow velocity measurement unit 101 is defined as V₁, and a blood flow velocity in the skin at a certain position on the rear head portion is defined as V₂.

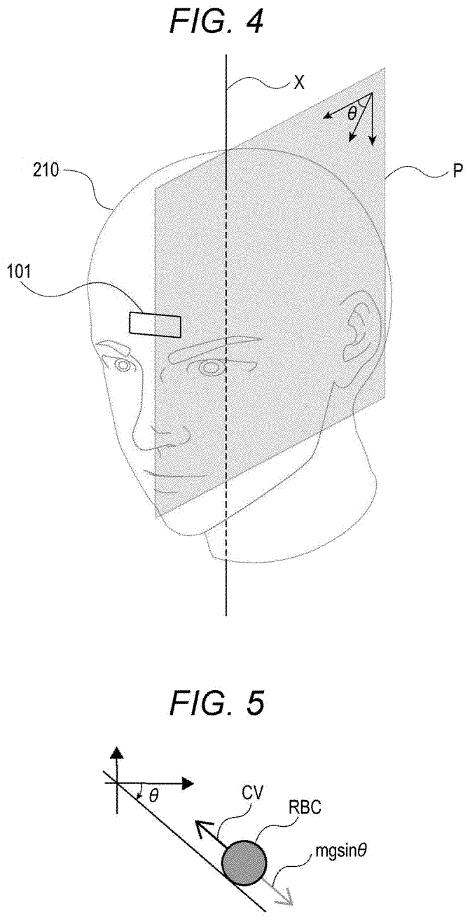

The state in which the person 210 faces downward by an angle θ is shown on the right side of FIG. 2. The angle θ is the amount of change in the inclination of the blood flow velocity measurement unit 101. As shown in FIG. 4, the angle θ is an angle projected onto a plane P passing the blood flow velocity measurement unit 101 and a center line X of the head of the person 210. As shown on the right side of FIG. 2, when the head faces downward by the angle θ, the blood flow velocity at the certain position of the frontal head portion measured by the blood flow velocity measurement unit 101 increases by a fluctuation amount v (that is V₁+v) and the blood flow velocity in the blood vessel at a certain position on the rear head portion decreases by the fluctuation amount v (that is, V₂−v).

The fluctuation amount v will be described below using a model related to resistance and gravity acting on a red blood cell of mass m as shown in FIG. 5. In FIG. 5, cv is a resistance acting on a red blood cell RBC, and the resistance is caused by, for example, a blood vessel. In FIG. 5, mg sin θ is a force generated by gravity acting on the red blood cell RBC. In the model, the fluctuation amount v is represented by the equation of motion of the following Formula (1).

$$\begin{matrix}[ Math . 1 ] & \\ m \frac{dv}{dt} = - cv + mg sin θ & ( 1 )\end{matrix}$$

The above Formula (1) is transformed into the following.

$$\begin{matrix}[ Math . 2 ] & \\ dv = - \frac{c}{m} ( v + \frac{mg}{c} sin θ ) dt & ( 2 )\end{matrix}$$

Assuming that c/m in Formula (2) is τ and mg/c is α, Formula (2) becomes Formula (3) below.

[Math. 3]

dv=−τ(v+α(sin θ))dt (3)

Formula (3) becomes the following Formula (4) by integrating both sides.

[Math. 4]

v=α×sin θe^{-t/τ} (4)

As described above, the fluctuation amount v is represented by Formula (4).

As described above, according to one embodiment of the present technology, the correction information for canceling the increase in the blood flow velocity caused by lowering in the position of the head may be the fluctuation amount v represented by Formula (4). Furthermore, the correction information for canceling the decrease in the blood flow velocity caused by the raising in the position of the head may also be the fluctuation amount v represented by Formula (4).

That is, in a case where the position of the blood flow velocity measurement unit 101 becomes low, the increase in the blood flow velocity caused by a change in the position of the head is removed by subtracting the fluctuation amount v represented by Formula (4) from the measured blood flow velocity. In a case where the position of the blood flow velocity measurement unit 101 becomes high, the decrease in the blood flow velocity caused by a change in the position of the head can be compensated by adding the fluctuation amount v represented by Formula (4) to the measured blood flow velocity.

The symbol a in Formula (4) is called a position characteristic correction parameter. The position characteristic correction parameter may be set or updated on the basis of the relationship between the position of the blood flow velocity measurement unit and the blood flow velocity fluctuation amount. The setting or update can be performed by the position characteristic correction parameter setting unit 106. An example of how to set the position characteristic correction parameter will be described below.

The blood flow velocity measurement unit 101 is mounted on the forehead of the person. With the blood flow velocity measurement unit 101 mounted, the person changes the position of the head from the reference position to various positions. The various positions can include, for example, one or more, two or more, three or more, or four selected from the position in a case where the person faces downward, the position in a case where the person faces upward, the position in a case where the head is inclined to the right, and the position in a case where the head is inclined to the left. In addition to these positions, for example, the various positions may include the position in a case where the person faces downward and the head is inclined to the right and/or left, the position in a case where the person faces upward and the head is inclined to the right and/or left, and the like.

At each of the various positions, the blood flow velocity measurement unit 101 measures the blood flow velocity at the forehead, and the detection unit 102 measures the position change amount of the blood flow velocity measurement unit 101. The position characteristic correction parameter setting unit 106 can set and/or update the position characteristic correction parameter on the basis of the blood flow velocity and the position change amount measured at the various positions. That is, the position characteristic correction parameter setting unit 106 can set and/or update the position characteristic correction parameter on the basis of the relationship between the position change amount in a case where the position of the blood flow velocity measurement unit is changed to various different positions from the reference position and the blood flow velocity (particularly, blood flow velocity fluctuation amount) measured at each of the various different positions.

The blood flow velocity measurement by the blood flow velocity measurement unit 101 may be measured at a point of time when a predetermined time has elapsed after the change in the position of the head to each of the various positions. The blood flow velocity changes with time delay with respect to the change in the position of the head. That is, the change in the blood flow velocity caused by the change in the position of the head has transient characteristics. Therefore, as described above, by measuring the blood flow velocity at a point of time when a predetermined time has elapsed after the change in the position of the head, it is possible to measure a more accurate blood flow velocity after the change in the position of the head. The predetermined time can be, for example, 1 second to 60 seconds, particularly 3 seconds to 40 seconds, and more particularly 5 seconds to 30 seconds.

Alternatively, the position of the head may be changed so slowly that the transient characteristics are negligible. Blood flow velocity may be measured after the slow change in the position of the head. In a case where the position of the head is changed slowly in this way, the movement of the head does not have to be stopped at a specific position. For example, the head may slowly move for one turn, and blood flow velocity may be measured sequentially during the one turn.

The symbol θ in the Formula (4) is the amount of change in the inclination of the blood flow velocity measurement unit 101 and may be an angle projected onto the plane passing the blood flow velocity measurement unit 101 and the center line of the head of the person 210 as shown in FIG. 4. The symbol θ may be measured by the detection unit 102, for example, may be measured by a 3-axis acceleration sensor (not shown) included in the detection unit 102. The detection unit 102 may be arranged at the center of the frontal head portion 211 so as to change the position in the same manner as the position of the blood flow velocity measurement unit 101. For example, by fixing the positional relationship between the detection unit 102 and the blood flow velocity measurement unit 101 in the biological information acquisition device 100, the blood flow velocity measurement unit 101 and the detection unit 102 perform the same positional change. Therefore, the position change amount of the detection unit 102 can be regarded as the position change amount of the blood flow velocity measurement unit 101.

The symbol θ in Formula (4) may be determined using, for example, the following Formula (5) based on an output value of the 3-axis acceleration sensor.

$$\begin{matrix}[ Math . 5 ] & \\ θ = \frac{{a}_{1} {b}_{1} + {a}_{2} {b}_{2} + {a}_{3} {b}_{3}}{\sqrt{{a}_{1}^{2} + {a}_{2}^{2} + {a}_{3}^{2}} \sqrt{{b}_{1}^{2} + {b}_{2}^{2} + {b}_{3}^{2}}} & ( 5 )\end{matrix}$$

In the above Formula (5), a=[a₁, a₂, a₃] is an output value of the 3-axis acceleration sensor when the person 210 is facing the front as shown on the left side of FIG. 2, that is, an output value of the 3-axis acceleration sensor at the reference position. In Formula (5), b=[b₁, b₂, b₃] is an output value of the 3-axis acceleration sensor in a case where the head of the person 210 faces downward by the angle θ as shown on the right side of FIG. 2. In the present technology, the angle θ may be measured by a gyro sensor.

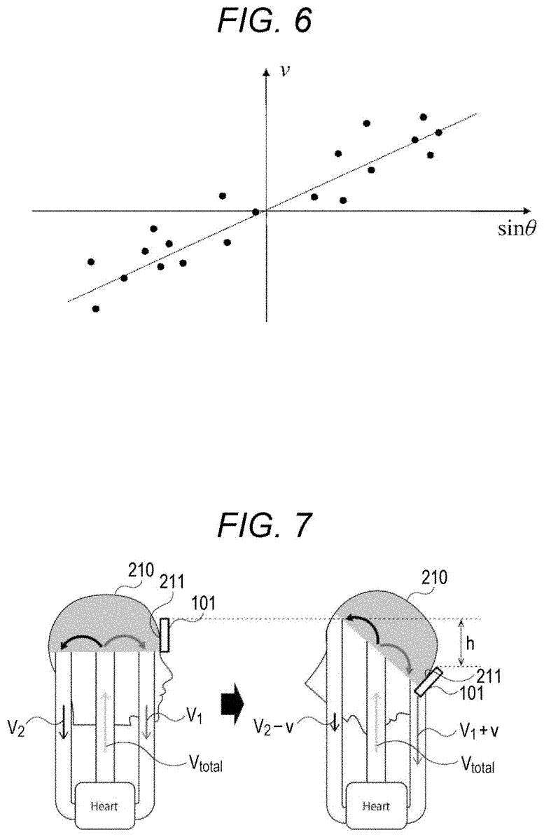

For example, at each of the position in a case where the person faces downward, the position in a case where the person faces upward, the position in a case where the head is inclined to the right, the position in a case where the head is inclined to the left, the position in a case where the person faces downward and the head is inclined to the right, the position in a case where the person faces downward and the head is inclined to the left, the position in a case where the person faces upward and the head is inclined to the right, and the position in a case where the person faces upward and the head is inclined to the left, the blood flow velocity fluctuation amount is measured by the blood flow velocity measurement device 201 and the position change amount is measured by the 3-axis acceleration sensor. These measurements at each position may be performed multiple times. The blood flow velocity fluctuation amount and the position change amount at each position are plotted on the vertical axis and the horizontal axis, respectively. The inclination of the straight line obtained by applying the least-squares method to the plotted data may be adopted as position characteristic correction parameter α. For example, by measurement of the blood flow velocity fluctuation amount and measurement of the position change amount at the various positions, it is possible to obtain plots and a straight line as shown in FIG. 6. In FIG. 6, the vertical axis is the blood flow fluctuation amount v, and the horizontal axis is the position change amount sine.

The symbol τ in the Formula (4) is called a transient characteristic correction parameter. The transient characteristic correction parameter is a parameter related to the transient characteristics of the blood flow velocity. As described above, the blood flow velocity changes with time delay with respect to the change in the position of the head, that is, the change in the blood flow velocity caused by the change in the position of the head has transient characteristics. In order to reflect the transient characteristics in a measurement value of the blood flow velocity, the above-mentioned transient characteristic correction parameter can be used.

The transient characteristic correction parameter τ may be calculated, for example, by fitting a blood flow velocity estimated value estimated by using the position characteristic correction parameter α to temporal blood flow velocity data temporally measured in the process of changing the position of the blood flow velocity measurement device 201 from the reference position to another position. The fitting may be performed by, for example, a linear fitting or may be performed by a non-linear fitting. For example, the transient characteristic correction parameter setting unit 107 can calculate the transient characteristic correction parameter as described above and set and/or update the parameter.

(2-2) Correction Information Based on the Amount of Change in Height

According to another embodiment of the present technology, the correction information generation unit can generate correction information of the blood flow velocity on the basis of the amount of change in the height of the blood flow velocity measurement unit. A more specific example of generating the correction information will be described below.

The change in the blood flow velocity caused by the change in the position of the head (the increase in the blood flow velocity and the decrease in the blood flow velocity) will be described below with reference to FIG. 7. FIG. 7 is a model diagram for explaining a change in the blood flow velocity. In FIG. 7, the change in the position of the blood flow velocity measurement unit 101 is indicated by using height h instead of the angle θ in FIG. 2. That is, the left side of FIG. 7 shows the state where the person 210 is facing the front, that is, the case where the blood flow velocity measurement unit 101 is in the reference position, and the right side of FIG. 7 shows the position of the head 211 lowered by h and the position of the blood flow velocity measurement unit 101 is also lowered by h accordingly.

On the left side of FIG. 7, the person 210 faces the front, and the position of the blood flow velocity measurement unit 101 in this state is used as a reference position. In the case shown on the left side of FIG. 7, for example, a blood flow flowing from the heart to the head is defined as Vₜₒₜₐₗ, a blood flow velocity at a certain position on the frontal head portion measured by the blood flow velocity measurement unit 101 is defined as V₁, and a blood flow velocity in a blood vessel at a certain position on the rear head portion is defined as V₂.

On the right side of FIG. 7, the height of the frontal head portion 211 of the person 210 is lowered by h, and the position of the blood flow velocity measurement unit 101 is lowered by the height h accordingly. As shown on the right side of FIG. 7, when the position of the head is lowered by the height h, the blood flow velocity at the certain position of the frontal head portion measured by the blood flow velocity measurement unit 101 increases by a fluctuation amount v (that is V₁+v) and the blood flow velocity in the blood vessel at a certain position on the rear head portion decreases by the fluctuation amount v (that is, V₂−v).

The fluctuation amount v can be represented by the equation of motion using mgh instead of mg sin θ in the above Formula (1). The following Formula (6) can be obtained by transforming the equation of motion in a manner similar to the transformation from the above Formula (1) to Formula (4).

[Math. 6]

v=α×he^{-t/τ} (6)

As described above, according to one embodiment of the present technology, the correction information for canceling the increase in the blood flow velocity caused by lowering in the position of the head may be the fluctuation amount v represented by Formula (6). Furthermore, the correction information for canceling the decrease in the blood flow velocity caused by the raising in the position of the head may also be the fluctuation amount v represented by Formula (6).

That is, in a case where the position of the blood flow velocity measurement unit 101 becomes low, the increase in the blood flow velocity caused by a change in the position of the head is removed by subtracting the fluctuation amount v represented by Formula (6) from the measured blood flow velocity. In a case where the position of the blood flow velocity measurement unit 101 becomes high, the decrease in the blood flow velocity caused by a change in the position of the head can be compensated by adding the fluctuation amount v represented by Formula (6) to the measured blood flow velocity.

The symbol a in Formula (6) is called a position characteristic correction parameter similar to α in Formula (4). The position characteristic correction parameter may be set on the basis of the relationship between the position of the blood flow velocity measurement unit and the blood flow velocity fluctuation amount. The position characteristic correction parameter setting unit 106 can set and/or update the position characteristic correction parameter on the basis of the blood flow velocity and the position change amount measured at specific various positions. That is, the position characteristic correction parameter setting unit 106 can set and/or update the position characteristic correction parameter on the basis of the relationship between the position change amount in a case where the position of the blood flow velocity measurement unit is changed to various different positions from the reference position and the blood flow velocity (particularly, blood flow velocity fluctuation amount) measured at each of the various different positions. An example of how to set the position characteristic correction parameter will be described below.

The blood flow velocity measurement unit 101 is mounted on the forehead of the person. With the blood flow velocity measurement unit 101 mounted, the person changes the position of the head from the reference position to various positions.

The various positions can include, for example, one or more, two or more, three or more, or four selected from the position in a case where the person faces downward, the position in a case where the person faces upward, the position in a case where the head is inclined to the right, and the position in a case where the head is inclined to the left. In addition to these positions, for example, the various positions may include the position in a case where the person faces downward and the head is inclined to the right and/or left, the position in a case where the person faces upward and the head is inclined to the right and/or left, and the like.

At each of the various positions, the blood flow velocity measurement unit 101 measures the blood flow velocity at the forehead, and the detection unit 102 detects the position change amount of the blood flow velocity measurement unit 101.

The blood flow velocity measurement by the blood flow velocity measurement unit 101 may be measured at a point of time when a predetermined time has elapsed after the change in the position of the head to each of the various positions. Therefore, as described above, it is possible to eliminate the transient characteristics and measure the more accurate blood flow velocity after the change in the head position. The predetermined time can be, for example, 1 second to 60 seconds, particularly 3 seconds to 40 seconds, and more particularly 5 seconds to 30 seconds.

Alternatively, the position of the head may be changed so slowly that the transient characteristics are negligible. Blood flow velocity may be measured after the slow change in the position of the head. In a case where the position of the head is changed slowly in this way, the movement of the head does not have to be stopped at a specific position.

The symbol h in Formula (6) can be the amount of change in the height of the blood flow velocity measurement unit 101. The symbol h may be measured by the detection unit 102, for example, by a barometric pressure sensor (not shown) included in the detection unit 102. The detection unit 102 may be arranged at the center of the frontal head portion 211 so as to change the position in the same manner as the position of the blood flow velocity measurement unit 101. For example, by fixing the positional relationship between the detection unit 102 and the blood flow velocity measurement unit 101 in the biological information acquisition device 100, the blood flow velocity measurement unit 101 and the detection unit 102 perform the same positional change. Therefore, the position change amount of the detection unit 102 can be regarded as the position change amount of the blood flow velocity measurement unit 101.

The symbol h in Formula (6) may be determined by, for example, an acceleration sensor, a gyro sensor, or a barometric pressure sensor. For example, for symbol h in

Formula (6), the amount of change in height h may be determined on the basis of the change in barometric pressure measured by the barometric pressure sensor.

For example, in each of cases where the head is at various heights, the blood flow velocity measurement unit 101 measures the blood flow velocity fluctuation amount and the detection unit 102 measures the position change amount. These measurements at each height may be performed multiple times. The blood flow velocity fluctuation amount and the position change amount at each height are plotted on the vertical axis and the horizontal axis, respectively. The inclination of the straight line obtained by applying the least-squares method to the plotted data may be adopted as position characteristic correction parameter α.

The symbol τ in Formula (6) is called a transient characteristic correction parameter similar to τ in Formula (4). The transient characteristic correction parameter is a parameter related to the transient characteristics of the blood flow velocity. As mentioned above, the change in the blood flow velocity caused by the change in the position of the head has transient characteristics. In order to reflect the transient characteristics in a measurement value of the blood flow velocity, the above-mentioned transient characteristic correction parameter can be used.

The transient characteristic correction parameter τ may be calculated, for example, by fitting a blood flow velocity estimated value estimated by using the position characteristic correction parameter α to temporal blood flow velocity data temporally measured in the process of changing the position of the blood flow velocity measurement unit 101 from the reference position to another position. The fitting may be performed by, for example, a linear fitting or may be performed by a non-linear fitting. For example, the transient characteristic correction parameter setting unit 107 can calculate the transient characteristic correction parameter as described above and set and/or update the parameter.

(3) Example of Configuration of the Biological Information Acquisition Device (Correction Based on Moving Velocity)

According to another embodiment of the present technology, the biological information acquisition device generates correction information on the basis of the moving velocity of the blood flow velocity measurement unit installed on a head, and then corrects the blood flow velocity measured by the blood flow velocity measurement unit by using the correction information. The moving velocity is preferably the angular velocity of the blood flow velocity measurement unit or the velocity of the blood flow velocity measurement unit, and more preferably the angular velocity of the blood flow velocity measurement unit.

An example of the biological information acquisition device in this embodiment will be described below with reference to FIG. 13. FIG. 13 is an example of a block diagram of the biological information acquisition device according to this embodiment.

As shown in FIG. 13, a biological information acquisition device 300 includes a blood flow velocity measurement unit 301, a detection unit 302, a processing unit 303, and an output unit 308. The processing unit 303 includes a correction information generation unit 304, a correction unit 305, a velocity characteristic correction parameter setting unit 309, and a transient characteristic correction parameter setting unit 307.

The blood flow velocity measurement unit 301 and the output unit 308 are the same as the blood flow velocity measurement unit 101 and the output unit 108 described in “(2) Example of configuration of the biological information acquisition device (correction based on position change amount)” above, and their descriptions also apply to the present embodiment.

The detection unit 302 detects the moving velocity of the blood flow velocity measurement unit 301. The moving velocity may be, for example, a velocity related to linear movement, or an angular velocity related to rotational movement. The detection unit 302 can include, for example, a gyro sensor. Alternatively, the detection unit 302 can include an acceleration sensor, a barometric pressure sensor, or a geomagnetic sensor. More specifically, the detection unit 302 can include, for example, a 6DOF (degree of freedom) inertial sensor. The inertial sensor is a sensor including both a triaxial gyro sensor and a triaxial acceleration sensor. For example, any of these sensors can be provided in the biological information acquisition device 300 so that the position change is the same as the position change of the blood flow velocity measurement unit 301. For example, the positional relationship between the blood flow velocity measurement unit 301 and the detection unit 302 can be fixed. Therefore, the moving velocity detected by the detection unit 302 can be treated as the moving velocity of the blood flow velocity measurement unit 301.

Alternatively, the detection unit 302 may be a combination of a marker indicating the position of the blood flow velocity measurement unit 301 and a marker recognition device that recognizes the marker. The marker may be attached to the blood flow velocity measurement unit 301, or may be attached to the biological information acquisition device 300 so that the movement is the same as the movement of the blood flow velocity measurement unit 301. The marker recognition device can include an imaging element. By processing a still image or a moving image obtained by the imaging element, the moving velocity of the marker can be measured. The moving velocity of the marker can be treated as the moving velocity of the blood flow velocity measurement unit 301.

The processing unit 303 can include a processor such as a CPU and a memory such as RAM and/or ROM. A program for causing the device to execute the biological information acquisition method according to the present technology, a velocity characteristic correction parameter and a transient characteristic correction parameter described below, a program for setting or updating these parameters, and the like can be stored in the memory. The function of the processing unit 303 can be realized by the processor.

The correction information generation unit 304 generates correction information on the basis of the moving velocity of the blood flow velocity measurement unit 301 detected by the detection unit 302. Since the correction information is generated on the basis of the moving velocity of the blood flow velocity measurement unit 301, it is suitable for canceling a change (increase/decrease) in the blood flow velocity caused by the moving velocity of the head.

That is, according to one embodiment of the present technology, the correction information generation unit 304 can generate correction information for canceling fluctuations (for example, increase or decrease) of the blood flow velocity caused by the moving velocity of the head. For example, the correction information generation unit 304 can generate correction information for canceling an increase in the blood flow velocity caused by rotational movement of the head or correction information for canceling a decrease in the blood flow velocity caused by linear movement of the head. More specifically, the correction information generation unit 304 can generate correction information on the basis of the moving velocity, the velocity characteristic correction parameter set on the basis of the relationship between the moving velocity of the blood flow velocity measurement unit and the blood flow velocity fluctuation amount and/or the transient characteristic correction parameter related to transient characteristics of the blood flow velocity. A more specific example of the generated correction information will be described in (3-1) below.

The correction unit 305 corrects the blood flow velocity measured by the blood flow velocity measurement unit 301 by using the correction information generated by the correction information generation unit 304. For example, in a case where the blood flow velocity is increased or decreased by the rotation of the head or the linear movement of the head, the correction unit 305 uses the correction information generated by the correction information generation unit 304 to subtract the increase from the measured blood flow velocity or add the decrease to the measured blood flow velocity. As described above, in the present technology, the correction unit 305 can cancel the fluctuation of the blood flow velocity caused by the movement of the head by using the correction information.

The velocity characteristic correction parameter setting unit 309 sets and/or updates the velocity characteristic correction parameter set on the basis of the relationship between the moving velocity of the blood flow velocity measurement unit and the blood flow velocity fluctuation amount.

The velocity characteristic correction parameter setting unit 309 can set and/or update the velocity characteristic correction parameter before, for example, performing correction by the correction unit. The biological information acquisition device 300 may have a predetermined velocity characteristic correction parameter in advance or may not have the velocity characteristic correction parameter in advance.

In a case where the biological information acquisition device 300 has a predetermined velocity characteristic correction parameter in advance, the velocity characteristic correction parameter setting unit 309 may update the velocity characteristic correction parameter before the blood flow velocity is corrected according to the present technology. In a case where the biological information acquisition device 300 has a predetermined velocity characteristic correction parameter in advance, the velocity characteristic correction parameter may not be updated. For example, the velocity characteristic correction parameter set at the time of shipment of the biological information acquisition device 300 may be used as it is, or the velocity characteristic correction parameter set when a certain user uses the biological information acquisition device 300 for the first time may be used as it is at the next use (when the biological information acquisition device or the blood flow velocity measurement device is mounted after the second time).

In a case where the biological information acquisition device 300 does not have a velocity characteristic correction parameter in advance, the velocity characteristic correction parameter setting unit 309 may set the velocity characteristic correction parameter before the blood flow velocity is corrected according to the present technology.

In this way, by setting or updating the velocity characteristic correction parameter before the blood flow velocity is corrected according to the present technology, the velocity characteristic correction parameter according to a target (particularly person) whose blood flow is measured by the biological information acquisition device according to the present technology can be set or updated. More accurate correction information can be generated by the set or updated velocity characteristic correction parameter, and then, more appropriate correction of blood flow velocity can be performed on the basis of the more accurate correction information.

While the biological information acquisition device according to the present technology is mounted on the target, the velocity characteristic correction parameter may be updated at predetermined time intervals or for each buffer unit. More specifically, the update may be performed, for example, every 3 minutes to 3 hours, particularly every 5 minutes to 2 hours, and more particularly every 10 minutes to 1 hour. The buffer unit refers to one sample unit of the blood flow velocity measured by the blood flow velocity measurement device.

For example, the predetermined number of times of measuring the blood flow velocity can be one unit, or the predetermined time interval in which the blood flow velocity is measured can be one unit. By updating the velocity characteristic correction parameter at predetermined time intervals or for each buffer unit in this way, for example, in a case where the blood flow velocity is acquired by the biological information acquisition device according to the present technology for a long period of time, a more appropriate velocity characteristic correction parameter enables more appropriate correction of blood flow velocity.

The setting and/or update of the velocity characteristic correction parameter will be described in more detail below in (3-1).

The transient characteristic correction parameter setting unit 307 sets and/or updates the transient characteristic correction parameter relating to the transient characteristics of the blood flow velocity.

The transient characteristic correction parameter setting unit 307 can set and/or update the transient characteristic correction parameter, for example, before the blood flow velocity measurement unit performs correction by the correction unit. The biological information acquisition device 300 may have a predetermined transient characteristic correction parameter in advance or may not have the transient characteristic correction parameter in advance.

In a case where the biological information acquisition device 300 has a predetermined transient characteristic correction parameter in advance, the transient characteristic correction parameter setting unit 307 may update the transient characteristic correction parameter before the blood flow velocity is corrected according to the present technology. In a case where the biological information acquisition device 300 has a predetermined transient characteristic correction parameter in advance, the transient characteristic correction parameter may not be updated. For example, the transient characteristic correction parameter set at the time of shipment of the biological information acquisition device 300 may be used as it is, or the transient characteristic correction parameter set when a certain user uses the biological information acquisition device 300 for the first time may be used as it is at the next use (when the biological information acquisition device or the blood flow velocity measurement device is mounted after the second time).

In a case where the biological information acquisition device 300 does not have a transient characteristic correction parameter in advance, the transient characteristic correction parameter setting unit 307 may set the transient characteristic correction parameter before the blood flow velocity is corrected according to the present technology.

In this way, by setting or updating the transient characteristic correction parameter before the blood flow velocity is corrected according to the present technology, the transient characteristic correction parameter according to a target (particularly person) to which the biological information acquisition device according to the present technology is applied can be set or updated. More accurate correction information can be generated by the set or updated transient characteristic correction parameter, and then, more appropriate correction of blood flow velocity can be performed on the basis of the more accurate correction information.

While the biological information acquisition device according to the present technology is mounted on the target, the transient characteristic correction parameter may be updated at predetermined time intervals or for each buffer unit. More specifically, the update may be performed, for example, every 3 minutes to 3 hours, particularly every 5 minutes to 2 hours, and more particularly every 10 minutes to 1 hour. Therefore, for example, in a case where the blood flow velocity is acquired by the biological information acquisition device according to the present technology for a long period of time, a more appropriate transient characteristic correction parameter enables more appropriate correction of blood flow velocity.

The setting and/or update of the transient characteristic correction parameter will be described in more detail below in (3-1).

The biological information acquisition processing by the biological information acquisition device 300 may be performed as described, for example, in 3. “(3) Example of the third embodiment (biological information acquisition method using moving velocity of head)” below.

(3-1) Correction Information Based on Angular Velocity

According to one embodiment of the present technology, the correction information generation unit 304 can generate the correction information of the blood flow velocity on the basis of the angular velocity of the blood flow velocity measurement unit. In this embodiment, the angular velocity can be an angular velocity of the blood flow velocity measurement unit with respect to the center of the head. The generation of the correction information will be described in more detail below.

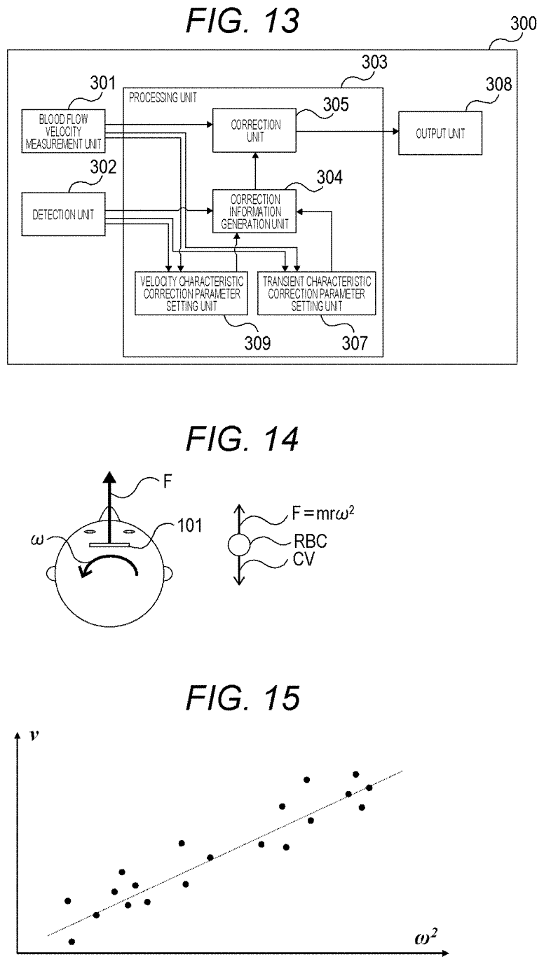

The velocity of blood flow flowing through a blood vessel at a certain position of the head changes according to rotational movement of the head. The fluctuation amount v of the blood flow velocity caused by the rotational movement of the head (the increase or decrease in the blood flow velocity) will be described below with reference to FIG. 14. FIG. 14 is a model diagram for explaining a change in the blood flow velocity.

As shown on the left side of FIG. 14, the blood flow velocity measurement unit 301 is installed on the frontal head portion (forehead) of a person. The blood flow velocity measurement unit 301 includes, for example, an LDF type blood flow velocity measurement device. As shown in FIG. 14, the blood flow velocity measurement unit 301 is arranged at the center of the frontal head portion. The blood flow velocity measurement unit 301 can measure the blood flow velocity of the frontal head portion by the LDF method.

On the left side of FIG. 14, the person faces upward in the drawing, and the position of the blood flow velocity measurement unit 301 in this state is used as a reference position. It is assumed that the head of the person rotates at an angular velocity co as shown on the left side of FIG. 14. A centrifugal force F is generated by the rotation, and the centrifugal force F acts on the blood flow velocity measurement unit 301, and further acts on the red blood cell RBC in the blood flow measured by the blood flow velocity measurement unit 301. The centrifugal force F is represented by F =mrω². Where m is the mass of the red blood cell RBC and r is the radius of the rotation, for example, the distance between the center of the rotation and the red blood cell RBC. In addition to the centrifugal force F, resistance cv acts on the red blood cell RBC. The resistance cv is caused by, for example, a blood vessel, as described above with reference to FIG. 5. In the model shown in FIG. 14, the fluctuation amount v is represented by the equation of motion of the following Formula (7).

$$\begin{matrix}[ Math . 7 ] & \\ m \frac{dv}{dt} = - cv + mr {ω}^{2} & ( 7 )\end{matrix}$$

The above Formula (7) is transformed into the following.

$$\begin{matrix}[ Math . 8 ] & \\ d v = - \frac{c}{m} ( v + \frac{mr}{c} {ω}^{2} ) dt & ( 8 )\end{matrix}$$

Assuming that c/m in Formula (8) is τ and mr/c is β, Formula (8) becomes Formula (9) below.

[Math. 9]

dv=−τ(v+βω²)dt (9)

Formula (9) becomes the following Formula (10) by integrating both sides.

[Math. 10]

v=β×ω²e^{-t/τ} (10)

As described above, the fluctuation amount v is represented by Formula (10).

As described above, according to one embodiment of the present technology, the correction information for canceling the fluctuation in the blood flow velocity caused by the angular velocity of the head may be the fluctuation amount v represented by Formula (10).

That is, in a case where the blood flow velocity measurement unit 301 rotates, the increase or decrease in the blood flow velocity caused by the rotation of the head is removed by subtracting or adding the fluctuation amount v represented by Formula (10) with respect to the measured blood flow velocity. For example, in a case where blood is collected at a position measured by the blood flow velocity measurement unit 301, the blood flow velocity can be decreased by the movement of the head facing left or right. Furthermore, in a case where blood is collected in a vicinity of the position measured by the blood flow velocity measurement unit 301, the blood flow velocity can be increased as the blood flows into the position by the movement of the head facing left or right.

The symbol β in Formula (10) is called a velocity characteristic correction parameter. The velocity characteristic correction parameter may be set or updated on the basis of the relationship between the moving velocity (for example, angular velocity or velocity) of the blood flow velocity measurement unit and the blood flow velocity fluctuation amount. The setting or update can be performed by the velocity characteristic correction parameter setting unit 309. An example of how to set the velocity characteristic correction parameter will be described below.

The blood flow velocity measurement unit 301 is mounted on the person forehead. With the blood flow velocity measurement unit 301 being mounted, the person moves the head up and down at various velocities. Each of the movements of the head at the various velocities may be, for example, a movement in which the head is reciprocated up and down once in a cycle of 1 second to 10 seconds.

In each of the cases where the head is moved at the various moving velocities, the blood flow velocity measurement unit 301 measures the blood flow velocity at the forehead, and the detection unit 302 measures the moving velocity (for example, the angular velocity and/or velocity) of the blood flow velocity measurement unit 301. The velocity characteristic correction parameter setting unit 309 can set and/or update the velocity characteristic correction parameter β on the basis of the relationship between the blood flow velocity measured at the various moving velocities or the fluctuation amount of the blood flow velocity and the moving velocity in a case where the blood flow velocity is measured.

The symbol co in Formula (10) is the angular velocity in the movement of the blood flow velocity measurement unit 301. The angular velocity can be, for example, the angular velocity in the rotation of the head, as shown on the left side of FIG. 14. Note that, in FIG. 14, the angular velocity co is the angular velocity of the rotational motion in a case where the head is rotated left and right, but the angular velocity co may be the angular velocity in another other rotational motion. For example, the angular velocity ω may be the angular velocity of the rotational motion in a case where the head is rotated up and down.

The symbol co may be measured by the detection unit 302, for example, by a gyro sensor (not shown) included in the detection unit 302. The detection unit 302 may be arranged at the center of the frontal head portion so as to move at the same moving velocity as the moving velocity of the blood flow velocity measurement unit 301. For example, by fixing the positional relationship between the detection unit 302 and the blood flow velocity measurement unit 301 in the biological information acquisition device 300, the blood flow velocity measurement unit 301 and the detection unit 302 move at the same moving velocity. Therefore, the moving velocity of the detection unit 302 can be treated as the moving velocity of the blood flow velocity measurement unit 301.

The determination of co in Formula (10) can be determined, for example, on the basis of the output value of the gyro sensor used. The determination of ω can be appropriately performed according to the type of gyro sensor. Furthermore, for determining ω, not only a gyro sensor but also, for example, an acceleration sensor, a barometric pressure sensor, a geomagnetic sensor, or an optical marker may be used.

For example, in each of the cases where the head is moved at the various moving velocities, the blood flow velocity measurement unit 301 measures the blood flow velocity at the forehead, and the detection unit 302 measures the moving velocity (for example, the angular velocity and/or velocity) of the blood flow velocity measurement unit 301. The blood flow velocity fluctuation amount and the moving velocity in each movement are plotted on the vertical axis and the horizontal axis, respectively. The inclination of the straight line obtained by applying the least-squares method to the plotted data may be adopted as velocity characteristic correction parameter β. For example, by measurement of the blood flow velocity fluctuation amount and measurement of the moving velocity in various movements, it is possible to obtain plots and a straight line as shown in FIG. 15. In FIG. 15, the vertical axis is the blood flow fluctuation amount v, and the horizontal axis is the position change amount ω².

The symbol τ in Formula (10) is called a transient characteristic correction parameter. The transient characteristic correction parameter is a parameter related to the transient characteristics of the blood flow velocity. The blood flow velocity in a case where the head moves can change with time delay with respect to the movement of the head. That is, the change in the blood flow velocity caused by the moving velocity of the head can have transient characteristics. Regarding the transient characteristics, in order to reflect the transient characteristics in a measurement value of the blood flow velocity, the above-mentioned transient characteristic correction parameter can be used.

The transient characteristic correction parameter τ may be calculated, for example, by fitting a blood flow velocity estimated value estimated by using the velocity characteristic correction parameter β to temporal blood flow velocity data temporally measured in a case where the blood flow velocity measurement device 301 starts movement. The fitting may be performed by, for example, a linear fitting or may be performed by a non-linear fitting. For example, the transient characteristic correction parameter setting unit 307 can calculate the transient characteristic correction parameter as described above and set and/or update the parameter.

(4) Example of Configuration of the Biological Information Acquisition Device (Correction Based on Position Change Amount and Moving Velocity)

According to yet another embodiment of the present technology, the biological information acquisition device generates correction information on the basis of the position change amount and the moving velocity of a blood flow velocity measurement unit installed on a head, and then corrects the blood flow velocity measured by the blood flow velocity measurement unit by using the correction information.

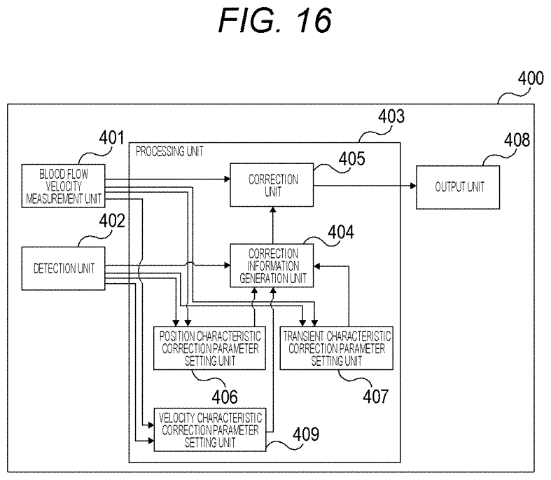

An example of the biological information acquisition device in this embodiment will be described below with reference to FIG. 16. FIG. 16 is an example of a block diagram of the biological information acquisition device of this embodiment.

As shown in FIG. 16, a biological information acquisition device 400 includes a blood flow velocity measurement unit 401, a detection unit 402, a processing unit 403, and an output unit 408. The processing unit 403 includes a correction information generation unit 404, a correction unit 405, a position characteristic correction parameter setting unit 406, a velocity characteristic correction parameter setting unit 409, and a transient characteristic correction parameter setting unit 407.

The blood flow velocity measurement unit 401 and the output unit 408 are the same as the blood flow velocity measurement unit 101 and the output unit 108 described in “(2) Example of configuration of the biological information acquisition device (correction based on position change amount)” above, and their descriptions also apply to the present embodiment.

The detection unit 402 detects the position change amount and the moving velocity of the blood flow velocity measurement unit 401. The position change amount is as described in “(2) Example of configuration of the biological information acquisition device (correction based on position change amount)” above, and its description also applies to the present embodiment.

Furthermore, the moving velocity is as described in “(3) Example of configuration of the biological information acquisition device (correction based on moving velocity)” above, and its description also applies to the present embodiment.

The processing unit 403 can include a processor such as a CPU and a memory such as RAM and/or ROM. A program for causing the device to execute the biological information acquisition method according to the present technology, a position characteristic correction parameter, a velocity characteristic correction parameter, and a transient characteristic correction parameter described below, a program for setting or updating these parameters, and the like can be stored in the memory. The function of the processing unit 403 can be realized by the processor.

The correction information generation unit 404 generates correction information on the basis of the position change amount and the moving velocity of the blood flow velocity measurement unit 401 detected by the detection unit 402. Since the correction information is generated on the basis of the position change amount and the moving velocity of the blood flow velocity measurement unit 401, it is suitable for canceling a change in the blood flow velocity caused by the position change amount of the head and a change (increase/decrease) in the blood flow velocity caused by the moving velocity of the head.

That is, according to one embodiment of the present technology, the correction information generation unit 404 can generate correction information for canceling fluctuations (for example, increase or decrease) of the blood flow velocity caused by the position change amount of the head and fluctuations (for example, increase or decrease) of the blood flow velocity caused by the moving velocity of the head. For example, the correction information generation unit 404 can generate both correction information for canceling the fluctuation of the blood flow velocity caused by the change in the position of the head and correction information for canceling the fluctuation of the blood flow velocity caused by the moving velocity of the head, and can add up these two pieces of correction information. The added-up correction information can be used in the correction by the correction unit 405 below. The added-up correction information is particularly suitable for eliminating the influence of the position change and the movement of the head from the measured blood flow velocity.

When obtaining the added-up correction information, the correction information generation unit 404 can weight each of the above two pieces of correction information. The coefficient for weighting may be determined according to the measurement target of the blood flow velocity, or may be preset.

Here, the correction information for canceling the fluctuation of the blood flow velocity caused by the change in the position of the head is as described in “(2) Example of configuration of the biological information acquisition device (correction based on position change amount)” above, and its description also applies to the present embodiment. Furthermore, the correction information for canceling the fluctuation of the blood flow velocity caused by the moving velocity of the head is as described in “(3) Example of configuration of the biological information acquisition device (correction based on moving velocity)” above, and its description also applies to the present embodiment.

The correction unit 405 corrects the blood flow velocity measured by the blood flow velocity measurement unit 401 by using the correction information generated by the correction information generation unit 404, particularly the added-up correction information. That is, the correction unit 405 can use the correction information to cancel the fluctuation (for example, increase or decrease) of the blood flow velocity caused by the position change of the head and the fluctuation (for example, increase or decrease) of the blood flow velocity caused by the moving velocity of the head. By this correction, the influence of the change in the position of the head and the moving velocity of the head is removed from the blood flow velocity measured by the blood flow velocity measurement unit 401.

The position characteristic correction parameter setting unit 406 is the same as the position characteristic correction parameter setting unit 106 described in “(2) Example of configuration of the biological information acquisition device (correction based on position change amount)” above, and its description also applies to the present embodiment.

The velocity characteristic correction parameter setting unit 409 is the same as the velocity characteristic correction parameter setting unit 309 described in “(3) Example of configuration of the biological information acquisition device (correction based on moving velocity)” above, and its description also applies to the present embodiment.

For the transient characteristic correction parameter setting unit 407, both descriptions of the transient characteristic correction parameter setting unit 107 and the transient characteristic correction parameter setting unit 307 described in “(2) Example of configuration of the biological information acquisition device (correction based on position change amount)” and “(3) Example of configuration of the biological information acquisition device (correction based on moving velocity)” apply.

The biological information acquisition processing by the biological information acquisition device 400 may be performed as described, for example, in 3. “(4) Example of the third embodiment (biological information acquisition method using position change amount and moving velocity of head)” below.

### 2. SECOND EMBODIMENT (HEAD-MOUNTED INFORMATION PRESENTATION DEVICE)

(1) Description of the Second Embodiment

The head-mounted information presentation device according to the present technology, similar to the biological information acquisition device described in “1. First embodiment (biological information acquisition device)” above, generates correction information on the basis of the position change amount of a blood flow velocity measurement unit installed on a head, the moving velocity of the blood flow velocity measurement unit, or both the position change amount and the moving velocity of the blood flow velocity measurement unit, and then corrects the blood flow velocity measured by the blood flow velocity measurement unit by using the correction information. Therefore, it is possible to remove fluctuations in the blood flow velocity caused by a change in the posture of a person and/or moving velocity, and thus it is possible to obtain blood flow velocity data that enables a more accurate grasp of the health state and/or the psychological state of the person.

In a case of using the head-mounted information presentation device for a long time, it may be desirable to temporally stop the use and take a break depending on the physical condition of the user. As described above, the head-mounted information presentation device according to the present technology can obtain blood flow velocity data that enables a more accurate grasp of the health state of the person. By more accurately grasping the health state of the user on the basis of the blood flow velocity data, it is possible to encourage the user to take a break at a more appropriate timing.

The head-mounted information presentation device according to the present technology can be a device for presenting information to the user while being mounted on the user's head. The information may be, for example, video information or sound information. For example, the device can be configured as a head-mounted display device (head-mounted display) or head-mounted headphones.

(2) Configuration of the Head-Mounted Information Presentation Device

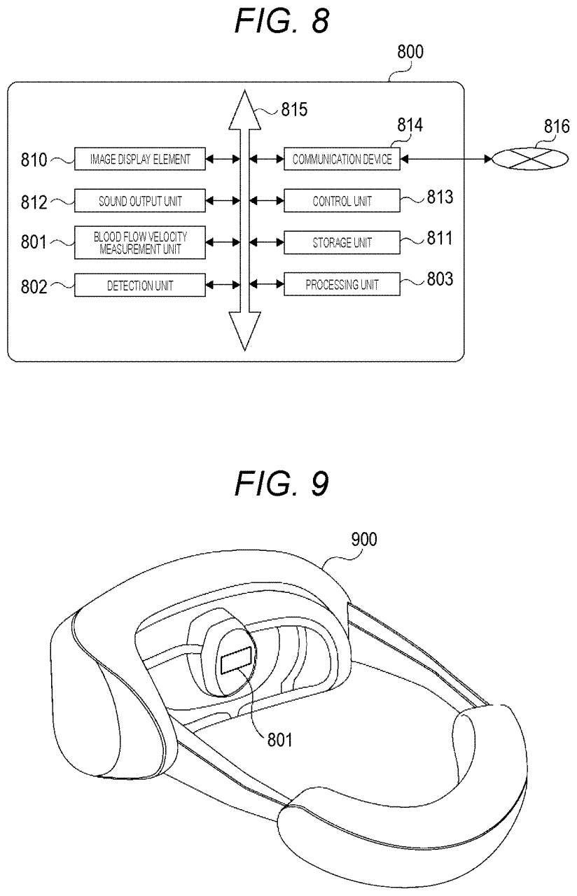

An example of the head-mounted information presentation device according to the present technology will be described below with reference to FIG. 8. FIG. 8 is an example of a configuration of a head-mounted display device according to the present technology.

As shown in FIG. 8, a head-mounted display device 800 includes a blood flow velocity measurement unit 801, a detection unit 802, and a processing unit 803. The blood flow velocity measurement unit 801, the detection unit 802, and the processing unit 803 of the head-mounted display device 800 may be the same as the blood flow velocity measurement unit 101, the detection unit 102, and the processing unit 103 included in the biological information acquisition device 100 described with reference to FIG. 1, may be the same as the blood flow velocity measurement unit 301, the detection unit 302, and the processing unit 303 included in the biological information acquisition device 300 described with reference to FIG. 13, or may be the same as the blood flow velocity measurement unit 401, the detection unit 402, and the processing unit 403 included in the biological information acquisition device 400 described with reference to FIG. 16. That is, the contents described with respect to the biological information acquisition devices 100, 300, and 400 also apply to these components in the head-mounted display device 800.

The processing unit 803 includes a correction information generation unit, a correction unit, a position characteristic correction parameter setting unit, and a transient characteristic correction parameter setting unit (none of which is shown). These are also the same as the correction information generation unit, the correction unit, the position characteristic correction parameter setting unit, and the velocity characteristic correction parameter setting unit, and the transient characteristic correction parameter setting unit included in the biological information acquisition device 100, 300, or 400 described with reference to FIG. 1, 13, or 16, and the contents described regarding the biological information acquisition devices 100, 300, and 400 also apply to these components in the head-mounted display device 800.

With these components in the head-mounted display device 800, it is possible to acquire the blood flow velocity from which the fluctuation in the blood flow velocity caused by the change in the posture of the person and/or the moving velocity has been removed.

The head-mounted display device 800 further includes an image display element 810, a storage unit 811 and a sound output unit (for example, a speaker) 812, a control unit 813, and a communication device 814. As these components, those known in the art may be adopted.

The blood flow velocity measurement unit 801, the detection unit 802, the processing unit 803, the image display element 810, the storage unit 811, the sound output unit 812, the control unit 813, and the communication device 814 are connected via a bus 815.

The head-mounted display device 800 can present an image to the user through the image display element 810 on the basis of image data stored in the storage unit 811. Furthermore, the head-mounted display device 800 can cause a sound to reach the user through the sound output unit 812 on the basis of sound data stored in the storage unit 811. The control unit 813 can control the image display element 810 and the sound output unit 812 to present the image to the user or cause the sound to reach the user as described above. The image data and the sound data may be acquired from the outside of the head-mounted display device 800 by the communication device 814 via a network 816 by wire or wirelessly.

The image display element 810 can include a liquid crystal display element (LCD) and an optical system. The image display element 810 may be configured to present an image formed by the liquid crystal display element to the user via the optical system. As the LCD and the optical system constituting the image display element 810, those known in the art may be adopted.

The storage unit 811 may include RAM, ROM, and other semiconductor memories. The storage unit 811 can store programs used by the control unit 813, for example, a program for controlling the image display element 810, a program for controlling the sound output unit 812, and the like. The storage unit 811 may store the image data and sound data described above. The storage unit 811 may store image data or sound data for presenting the blood flow velocity to the user, or information presented to the user on the basis of the blood flow velocity (for example, a video or sound for encouraging to take a break).

The control unit 813 can include a CPU or an MPU. The control unit 813 executes a function for displaying an image or presenting a sound according to a program stored in the storage unit 811.

The communication device 814 connects the head-mounted display device 800 to the network 816 by wire or wirelessly. The communication device 814 can acquire various data (for example, image data or sound data) via the network 816. The acquired data can be stored in, for example, the storage unit 811.

The head-mounted display device according to the present technology may be configured as a device 900 for presenting a virtual reality (VR) video to a user as shown in FIG. 9, for example.

For example, in a case where the user wears the head-mounted display device 900 on the head and watches a video presented from the device, the biological information acquisition processing according to the present technology can be performed periodically or continuously. The measurement of a blood flow velocity used for the biological information acquisition processing according to the present technology is performed by the blood flow velocity measurement unit 801. The blood flow velocity measurement unit 801 is configured to be able to measure the blood flow velocity of the frontal head portion. The biological information acquisition processing as described, for example, in “1. First embodiment (biological information acquisition device)” above may be performed by the blood flow velocity measurement unit 801, and the detection unit 802 and the processing unit 803, which are built into the head-mounted display device 900.

By the biological information acquisition processing, the blood flow velocity from which the fluctuation caused by the change in the position of the head and/or the moving velocity of the head has been removed is periodically or continuously acquired. In a case where the acquired blood flow velocity falls within a predetermined numerical range, for example, the control unit 813 can present a video or sound for encouraging the user to take a break through the image display element 810 or the sound output unit 812 to the user. Therefore, it is possible to encourage the user to take a break at an appropriate timing.

### 3. THIRD EMBODIMENT (BIOLOGICAL INFORMATION ACQUISITION METHOD)

(1) Description of the Third Embodiment

The present technology also provides a biological information acquisition method including: a blood flow velocity measurement process of measuring the blood flow velocity of a head by a blood flow velocity measurement device installed on the head, a detection process of detecting a position change amount of the blood flow velocity measurement device, a moving velocity of the blood flow velocity measurement device, or both the position change amount and the moving velocity of the blood flow velocity measurement device, a correction information generation process of generating correction information on the basis of the position change amount, the moving velocity, or the change amount and the moving velocity, and a correction process of correcting the blood flow velocity measured by the blood flow velocity measurement device using the correction information. By the biological information acquisition method, from the measured blood flow velocity of the head, the fluctuations in the blood flow velocity caused by the change in the position of the head and/or the moving velocity of the head, i.e., fluctuations in the blood flow velocity caused by the change in the posture of the person or the movement of the person can be removed.

(2) Example of the Third Embodiment (Biological Information Acquisition Method Using Position Change Amount of Head)

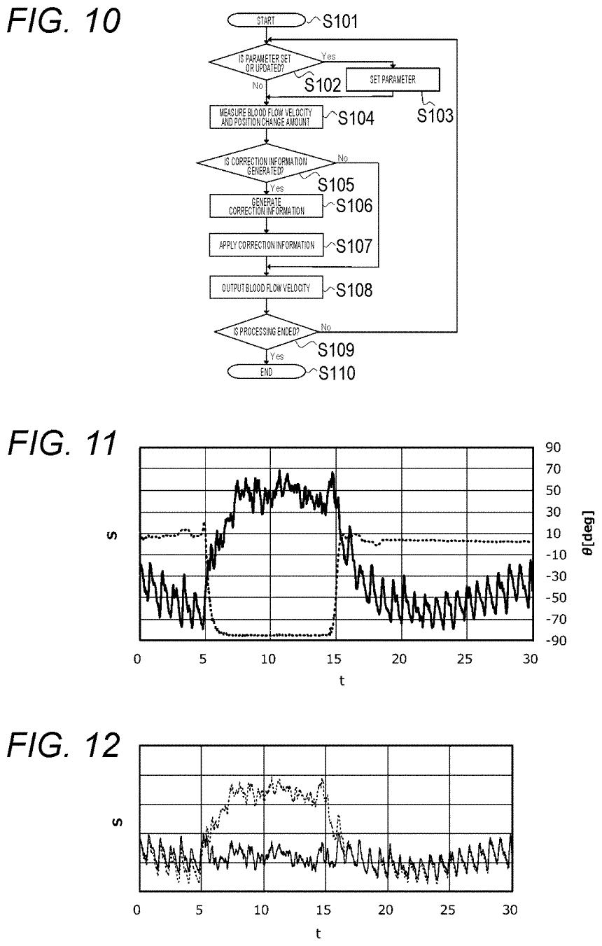

An example of the biological information acquisition method according to the present technology will be described below with reference to FIGS. 1 and 10. FIG. 1 is as described above. FIG. 10 is an example of a flow chart of the biological information acquisition processing.

In step S101 of FIG. 10, the biological information acquisition device 100 starts the biological information acquisition processing according to the present technology.

In step S102, the processing unit 103 determines whether to set the parameter in the formula used to correct the blood flow velocity. For example, the processing unit 103 can present a video asking the user of the biological information acquisition device 100 whether to set or update the parameter through the output unit 108. According to the user's selection to set or update the parameter, the processing unit 103 advances the processing to step S103. According to the user's selection not to set or update the parameter, the processing unit 103 advances the processing to step S104.

The parameter can be the position characteristic correction parameter and/or the transient characteristic correction parameter described above.

The video may be used to ask, for example, whether the user has been changed or whether to optimize a blood flow velocity measurement method for the user. The processing may proceed to step S103 according to the fact that the user has been changed or the user has selected optimization of the measurement method.

In step S103, the processing unit 103, particularly the position characteristic correction parameter setting unit 106 and/or the transient characteristic correction parameter setting unit 107 included in the processing unit 103, sets or updates the position characteristic correction parameter and/or the transient characteristic correction parameter. The setting or update of these parameters may be performed, for example, as described in “(1) Description of the first embodiment” above.

In step S104, the processing unit 103 drives the blood flow velocity measurement unit 101 and the detection unit 102 to measure the blood flow velocity of the head of the user on which the biological information acquisition device 100 is mounted and the position change amount of the blood flow velocity measurement unit 101.

In step S105, the processing unit 103 determines whether to generate correction information. The determination may be performed depending on whether the position change amount exceeding a predetermined threshold value has been measured, for example, in step S104.

For example, in a case where the position change amount measured in step S104 is equal to or greater than the predetermined threshold value, the processing unit 103 determines to generate the correction information. The processing unit 103 advances the processing to step S106 according to the determination that the correction information is generated. The processing unit 103 advances the processing to step S108 according to the determination that the correction information is not generated.

In step S106, the correction information generation unit 104 of the processing unit 103 generates the correction information of the blood flow velocity. The correction information is generated on the basis of the position change amount. The correction information may be generated, for example, as described in “1. First embodiment (biological information acquisition device)” above.

In step S107, the correction unit 105 of the processing unit 103 corrects the blood flow velocity measured in step S104 by using the correction information generated in step S106.

For example, in a case where the blood flow velocity has increased due to lowering in the position of the head, the correction unit 105 subtracts the increase from the measured blood flow velocity by using the correction information generated by the correction information generation unit 104. Alternatively, in a case where the blood flow velocity has decreased due to raising in the position of the head, the correction unit 105 adds the decrease to the measured blood flow velocity by using the correction information generated by the correction information generation unit 104.

In step S108, the processing unit 103 causes the output unit 108 to output the corrected blood flow velocity or uncorrected blood flow velocity.

For example, in a case where the output unit 108 includes a display, the blood flow velocity corrected or uncorrected by the display may be presented to the user.

Alternatively, the processing unit 103 may cause the output unit 108 to output advice according to the blood flow velocity. For example, in a case where the blood flow velocity is equal to or greater than the predetermined threshold value, a video encouraging the user to take a break can be displayed on the display by the processing unit 103.

In step S109, the processing unit 103 determines whether to end the biological information acquisition processing. For example, the processing unit 103 determines whether a predetermined time has elapsed from the start of the processing, determines to end the processing in a case where the predetermined time has elapsed, and determines not to end the processing in a case where the predetermined time has not elapsed. In a case where the processing unit 103 determines to end the biological information acquisition processing, the processing unit 103 advances the processing to step S110. In a case where the processing unit 103 determines not to end the biological information acquisition processing, the processing unit 103 returns the processing to step S102.

In step S110, the biological information acquisition device 100 ends the biological information acquisition processing according to the present technology.

(3) Example of the Third Embodiment (Biological Information Acquisition Method Using Moving Velocity of Head)

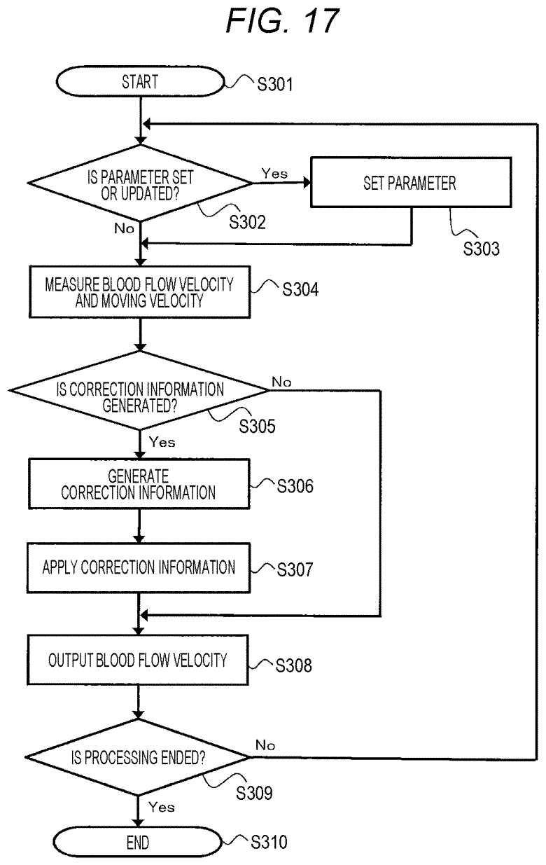

An example of the biological information acquisition method according to the present technology will be described below with reference to FIGS. 13 and 17. FIG. 13 is as described above. FIG. 17 is an example of a flow chart of the biological information acquisition processing.

In step S301 of FIG. 17, the biological information acquisition device 300 starts the biological information acquisition processing according to the present technology.

In step S302, the processing unit 303 determines whether to set the parameter in the formula used to correct the blood flow velocity. For example, the processing unit 303 can present a video asking the user of the biological information acquisition device 300 whether to set or update the parameter through the output unit 308. According to the user's selection to set or update the parameter, the processing unit 303 advances the processing to step S303. According to the user's selection not to set or update the parameter, the processing unit 303 advances the processing to step S304.

The parameter can be the velocity characteristic correction parameter and/or the transient characteristic correction parameter described above.

The video may be used to ask, for example, whether the user has been changed or whether to optimize a blood flow velocity measurement method for the user. The processing may proceed to step S303 according to the fact that the user has been changed or the user has selected optimization of the measurement method.

In step S303, the processing unit 303, particularly the velocity characteristic correction parameter setting unit 309 and/or the transient characteristic correction parameter setting unit 307 included in the processing unit 303, sets or updates the velocity characteristic correction parameter and/or the transient characteristic correction parameter. The setting or update of these parameters may be performed, for example, as described in “(3) Example of configuration of the biological information acquisition device (correction based on moving velocity of head)” above.

In step S304, the processing unit 303 drives the blood flow velocity measurement unit 301 and the detection unit 302 to detect the blood flow velocity of the head of the user on which the biological information acquisition device 300 is mounted and the moving velocity of the blood flow velocity measurement unit 301.

In step S305, the processing unit 303 determines whether to generate correction information. The determination may be performed depending on whether the moving velocity exceeding a predetermined threshold value has been measured, for example, in step S304.

For example, in a case where the moving velocity (e.g., angular velocity) measured in step S304 is equal to or greater than the predetermined threshold value, the processing unit 303 determines to generate the correction information. The processing unit 303 advances the processing to step S306 according to the determination that the correction information is generated. The processing unit 303 advances the processing to step S308 according to the determination that the correction information is not generated.

In step S306, the correction information generation unit 304 of the processing unit 303 generates the correction information of the blood flow velocity. The correction information is generated on the basis of the moving velocity. The correction information may be generated, for example, as described in “1. First embodiment (biological information acquisition device)” above.

In step S307, the correction unit 305 of the processing unit 303 corrects the blood flow velocity measured in step S304 by using the correction information generated in step S306.

For example, in a case where the blood flow velocity has increased due to the movement of the head, the correction unit 305 subtracts the increase from the measured blood flow velocity by using the correction information generated by the correction information generation unit 304. Alternatively, in a case where the blood flow velocity has decreased due to the movement of the head, the correction unit 305 adds the decrease to the measured blood flow velocity by using the correction information generated by the correction information generation unit 304.

Since steps S308, S309, and S310 are the same as steps S108, S109, and S110 described above, respectively, the description thereof will be omitted.

(4) Example of the Third Embodiment (Biological Information Acquisition Method Using Position Change Amount and Moving Velocity of Head)

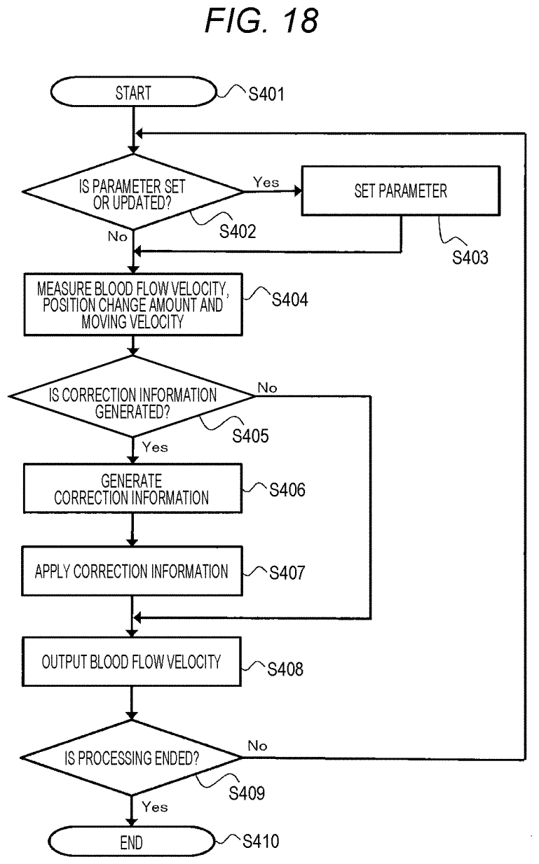

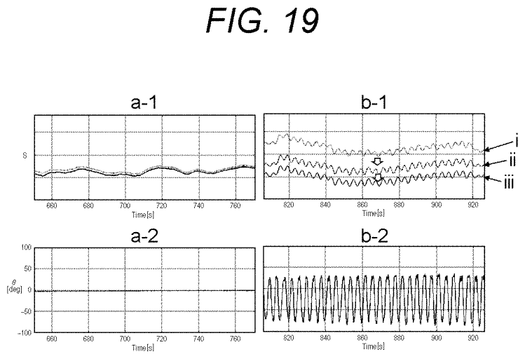

An example of the biological information acquisition method according to the present technology will be described below with reference to FIGS. 16 and 18. FIG. 16 is as described above. FIG. 18 is an example of a flow chart of the biological information acquisition processing.

In step S401 of FIG. 18, the biological information acquisition device 400 starts the biological information acquisition processing according to the present technology.

In step S402, the processing unit 403 determines whether to set the parameter in the formula used to correct the blood flow velocity. For example, the processing unit 403 can present a video asking the user of the biological information acquisition device 400 whether to set or update the parameter through the output unit 408. According to the user's selection to set or update the parameter, the processing unit 403 advances the processing to step S403. According to the user's selection not to set or update the parameter, the processing unit 403 advances the processing to step S404.

The parameter can be any one or two of the position characteristic correction parameter, the velocity characteristic correction parameter, and the transient characteristic correction parameter described above, or all of these three parameters.

The video may be used to ask, for example, whether the user has been changed or whether to optimize a blood flow velocity measurement method for the user. The processing may proceed to step S403 according to the fact that the user has been changed or the user has selected optimization of the measurement method.

In step S403, the processing unit 403, particularly the position characteristic correction parameter setting unit 406, the velocity characteristic correction parameter 409, or the transient characteristic correction parameter setting unit 407 included in the processing unit 403 sets or updates the position characteristic correction parameter, the velocity characteristic correction parameter, or the transient characteristic correction parameter. The setting or update of these parameters may be performed, for example, as described in “1. First embodiment (biological information acquisition device)” above.

In step S404, the processing unit 403 drives the blood flow velocity measurement unit 401 and the detection unit 402 to measure the blood flow velocity of the head of the user on which the biological information acquisition device 400 is mounted and the position change amount and/or the moving velocity of the blood flow velocity measurement unit 401.

In step S405, the processing unit 403 determines whether to generate correction information. The determination may be performed depending on whether the position change amount exceeding a predetermined threshold value and/or the moving velocity exceeding a predetermined threshold value have been measured, for example, in step S404.

For example, in a case where either one of the position change amount and the moving velocity measured in step S404 is equal to or greater than the predetermined threshold value, the processing unit 403 determines to generate the correction information. The processing unit 403 advances the processing to step S406 according to the determination that the correction information is generated. The processing unit 403 advances the processing to step S408 according to the determination that the correction information is not generated.

In step S406, the correction information generation unit 404 of the processing unit 403 generates the correction information of the blood flow velocity. The correction information is generated on the basis of the position change amount and the moving velocity. The correction information may be generated, for example, as described in “1. First embodiment (biological information acquisition device)” above.

In step S407, the correction unit 405 of the processing unit 403 corrects the blood flow velocity measured in step S404 by using the correction information generated in step S406.

Since steps S408, S409, and S410 are the same as steps S108, S109, and S110 described above, respectively, the description thereof will be omitted.

### 4. EXAMPLE

(1) Correction Based on Position Change Amount

An LDF type blood flow velocity measurement device and a 3-axis acceleration sensor were installed at the center of the forehead of a subject (person). The positional relationship between the blood flow velocity measurement device and the 3-axis acceleration sensor was fixed. The subject first faced the front for 5 seconds, then faced downward for 10 seconds, and then faced the front for 10 seconds. For a total of 25 seconds, the blood flow velocity at the forehead was continuously measured by the blood flow velocity measurement device, and the output value of the 3-axis acceleration sensor was continuously obtained. By using the output value of the 3-axis acceleration sensor, an inclination θ was calculated by the above Formula (5) and used as the inclination of the head of the subject. As shown in FIG. 4, the inclination θ is an angle projected onto a plane passing the blood flow velocity measurement device and the center line of the head. The relationship between the measured blood flow velocity and the inclination θ is shown in FIG. 11. In FIG. 11, the scale on the left axis is blood flow velocity S (unit: a.u.), the scale on the right axis is inclination θ (unit: degrees), and the horizontal axis is time t (unit: seconds). In FIG. 11, the dotted line is the inclination θ of the head, and the solid line is the measured blood flow velocity.

As shown in FIG. 11, the blood flow velocity between 5 and 15 seconds with the head facing downward was higher than the blood flow velocity at other times.

Similar to the above, the subject changed its posture again by facing the front for 5 seconds, then facing downward for 10 seconds, and then facing the front for 10 seconds. Then, similar to the above, the blood flow velocity at the forehead was measured and the inclination θ of the head was calculated. Correction was performed to subtract the fluctuation amount calculated according to the above Formula (4) from the measured blood flow velocity. Preliminarily calculated values were used as α and τ in Formula (4). The relationship between the blood flow velocity after correction and the inclination θ of the head is shown in FIG. 12. In FIG. 12, the scale on the vertical axis is blood flow velocity S (unit: a.u.), and the horizontal axis is time t (unit: seconds). In FIG. 12, the solid line is the blood flow velocity after correction, and the dotted line is the blood flow velocity before correction.

It can be seen from FIG. 12 that the blood flow velocity based on the inclination of the head could be removed by the above correction. Therefore, by the present technology, it is possible to remove the fluctuation amount of the blood flow velocity caused by a change in the position of the head from the measured blood flow velocity, and it is possible to obtain blood flow velocity data that enables a more accurate grasp of the health state and/or the psychological state of the person.

(2) Correction Based on Position Change Amount and Moving Velocity

An LDF type blood flow velocity measurement device and a 6DOF (degree of freedom) inertial sensor were installed at the center of the forehead of a subject (person). The inertial sensor is a sensor including both a triaxial gyro sensor and a triaxial acceleration sensor. The positional relationship between the blood flow velocity measurement device and the sensor was fixed.

The blood flow velocity and the position of the head of the subject at rest (state in which the position of the head did not change and the head did not move) were measured over a predetermined time. The measurement was performed by the blood flow velocity measurement device and the sensor. The results of these measurements are shown in a-1 and a-2 of FIG. 19, respectively. In FIG. 19, a-2 indicates that the position of the head is not changed. Furthermore, in FIG. 19, a-1 indicates that no sudden change in the blood flow velocity was observed.

Next, the subject continuously moved the head up and down once in a few seconds over several minutes.

During the movement of the head over the several minutes, the blood flow velocity and position of the head of the subject were measured by the blood flow velocity measurement device and the sensor. In FIG. 19, b-2 indicates that a continuous up-and-down movement of the position of the head was detected. In FIG. 19, i of b-1 is a blood flow velocity measured by the blood flow velocity measurement device. In FIG. 19, ii of b-1 is a blood flow velocity obtained by applying correction information based on the position change amount of the head to the blood flow velocity of i, and iii of b-1 of FIG. 19 is a blood flow velocity obtained by applying correction information based on the position change amount and the moving velocity of the head to the blood flow velocity of i.

From these results, in a case where the head moves, the blood flow velocity that is not corrected is higher than that at rest. It can be seen that by applying the correction information based on the position change amount to the blood flow velocity, it is possible to correct the blood flow velocity to be close to that at rest, and moreover, also by applying the correction information based on the position change amount and the moving velocity, it is possible to correct the blood flow velocity to be close to that at rest.

Note that the present technology may adopt the configuration described below.
[1] A biological information acquisition device including:

a blood flow velocity measurement unit that is installed on a head;

a detection unit that detects a position change amount of the blood flow velocity measurement unit, a moving velocity of the blood flow velocity measurement unit, or the position change amount and the moving velocity of the blood flow velocity measurement unit;

a correction information generation unit that generates correction information of a blood flow velocity on the basis of the position change amount, the moving velocity, or the position change amount and the moving velocity; and

a correction unit that corrects the blood flow velocity measured by the blood flow velocity measurement unit using the correction information.
[2] The biological information acquisition device according to [1], in which the position change amount includes an amount of change in inclination of the blood flow velocity measurement unit or an amount of change in height of the blood flow velocity measurement unit.
[3] The biological information acquisition device according to [1] or [2], in which the position change amount includes an amount of change in inclination of the blood flow velocity measurement unit, and the inclination includes an angle projected onto a plane passing the blood flow velocity measurement unit and a center line of the head.
[4] The biological information acquisition device according to any one of [1] to [3], in which the moving velocity includes an angular velocity of the blood flow velocity measurement unit or a velocity of the blood flow velocity measurement unit.
[5] The biological information acquisition device according to any one of [1] to [4], in which the moving velocity includes an angular velocity of the blood flow velocity measurement unit.
[6] The biological information acquisition device according to any one of [1] to [5], in which the correction information generation unit generates

correction information for canceling a fluctuation in blood flow velocity caused by a change in position of the head,

correction information for canceling a fluctuation in blood flow velocity caused by the moving velocity of the head, or

correction information obtained by adding up the two pieces of correction information.
[7] The biological information acquisition device according to any one of [1] to [6], in which the correction information generation unit generates

correction information on the basis of the position change amount, a position characteristic correction parameter set on the basis of a relationship between a position of the blood flow velocity measurement unit and a blood flow velocity fluctuation amount, and/or a transient characteristic correction parameter related to transient characteristics of the blood flow velocity, and/or generates

correction information on the basis of the moving velocity, a velocity characteristic correction parameter set on the basis of a relationship between the moving velocity of the blood flow velocity measurement unit and the blood flow velocity fluctuation amount, and/or the transient characteristic correction parameter related to the transient characteristics of the blood flow velocity.
[8] The biological information acquisition device according to [7], in which the biological information acquisition device further includes a position characteristic correction parameter setting unit that sets and/or updates the position characteristic correction parameter.
[9] The biological information acquisition device according to [8], in which the position characteristic correction parameter setting unit sets and/or updates the position characteristic correction parameter before correction by the correction unit is performed.
[10] The biological information acquisition device according to [8] or [9], in which the position characteristic correction parameter setting unit sets and/or updates the position characteristic correction parameter on the basis of a relationship between a position change amount in a case where the position of the blood flow velocity measurement unit is changed to various different positions from a reference position and a blood flow velocity measured at each of the various different positions.
[11] The biological information acquisition device according to [7], in which the biological information acquisition device further includes a velocity characteristic correction parameter setting unit that sets and/or updates the velocity characteristic correction parameter.
[12] The biological information acquisition device according to [11], in which the velocity characteristic correction parameter setting unit sets and/or updates the velocity characteristic correction parameter before correction by the correction unit is performed.
[13] The biological information acquisition device according to [11] or [12], in which the velocity characteristic correction parameter setting unit sets and/or updates the velocity characteristic correction parameter on the basis of a relationship between various moving velocities of the blood flow velocity measurement unit and a blood flow velocity measured at each of the various moving velocities.
[14] The biological information acquisition device according to [7], in which the biological information acquisition device further includes a transient characteristic correction parameter setting unit that sets and/or updates the transient characteristic correction parameter.
[15] The biological information acquisition device according to [14], in which the transient characteristic correction parameter setting unit sets and/or updates the transient characteristic correction parameter before correction by the correction unit is performed.
[16] The biological information acquisition device according to [14] or [15], in which the transient characteristic correction parameter setting unit sets and/or updates the transient characteristic correction parameter by fitting a blood flow velocity estimated value estimated by using the position characteristic correction parameter to temporal blood flow velocity data temporally measured in a process of changing the position of the blood flow velocity measurement unit to another position from a reference position.
[17] The biological information acquisition device according to any one of [1] to [16], in which the correction unit uses the correction information to cancel a fluctuation in blood flow velocity caused by a change in position of the head and/or a fluctuation in blood flow velocity caused by a moving velocity of the head.
[18] A head-mounted information presentation device including:

a blood flow velocity measurement unit that is installed on a head;

a detection unit that detects a position change amount of the blood flow velocity measurement unit, a moving velocity of the blood flow velocity measurement unit, or the position change amount and the moving velocity of the blood flow velocity measurement unit;

a correction information generation unit that generates correction information on the basis of the position change amount, the moving velocity, or the change amount and the moving velocity; and

a correction unit that corrects the blood flow velocity measured by the blood flow velocity measurement unit using the correction information.
[19] A biological information acquisition method including:

a blood flow velocity measurement process of measuring a blood flow velocity of a head using a blood flow velocity measurement device installed on the head;

a detection process of detecting a position change amount of the blood flow velocity measurement device, a moving velocity of the blood flow velocity measurement device, or the position change amount and the moving velocity of the blood flow velocity measurement device;

a correction information generation process of generating correction information on the basis of the position change amount, the moving velocity, or the change amount and the moving velocity; and

a correction process of correcting the blood flow velocity measured by the blood flow velocity measurement device using the correction information.

### REFERENCE SIGNS LIST

100 Biological information acquisition device
101 Blood flow velocity measurement unit
102 Detection unit
103 Processing unit
104 Correction information generation unit
105 Correction unit
106 Position characteristic correction parameter setting unit
107 Transient characteristic correction parameter setting unit
108 Output unit

1. A biological information acquisition device comprising:
a blood flow velocity measurement unit that is installed on a head;
a detection unit that detects a position change amount of the blood flow velocity measurement unit, a moving velocity of the blood flow velocity measurement unit, or the position change amount and the moving velocity of the blood flow velocity measurement unit;
a correction information generation unit that generates correction information of a blood flow velocity on a basis of the position change amount, the moving velocity, or the position change amount and the moving velocity; and
a correction unit that corrects the blood flow velocity measured by the blood flow velocity measurement unit using the correction information.

2. The biological information acquisition device according to claim 1, wherein the position change amount includes an amount of change in inclination of the blood flow velocity measurement unit or an amount of change in height of the blood flow velocity measurement unit.

3. The biological information acquisition device according to claim 1, wherein the position change amount includes an amount of change in inclination of the blood flow velocity measurement unit, and the inclination includes an angle projected onto a plane passing the blood flow velocity measurement unit and a center line of the head.

4. The biological information acquisition device according to claim 1, wherein the moving velocity includes an angular velocity of the blood flow velocity measurement unit or a velocity of the blood flow velocity measurement unit.

5. The biological information acquisition device according to claim 1, wherein the moving velocity includes an angular velocity of the blood flow velocity measurement unit.

6. The biological information acquisition device according to claim 1, wherein the correction information generation unit generates
correction information for canceling a fluctuation in blood flow velocity caused by a change in position of the head,
correction information for canceling a fluctuation in blood flow velocity caused by the moving velocity of the head, or
correction information obtained by adding up the two pieces of correction information.

7. The biological information acquisition device according to claim 1, wherein the correction information generation unit generates
correction information on a basis of the position change amount, a position characteristic correction parameter set on a basis of a relationship between a position of the blood flow velocity measurement unit and a blood flow velocity fluctuation amount, and/or a transient characteristic correction parameter related to transient characteristics of the blood flow velocity, and/or generates
correction information on a basis of the moving velocity, a velocity characteristic correction parameter set on a basis of a relationship between the moving velocity of the blood flow velocity measurement unit and the blood flow velocity fluctuation amount, and/or the transient characteristic correction parameter related to the transient characteristics of the blood flow velocity.

8. The biological information acquisition device according to claim 7, wherein the biological information acquisition device further comprises a position characteristic correction parameter setting unit that sets and/or updates the position characteristic correction parameter.

9. The biological information acquisition device according to claim 8, wherein the position characteristic correction parameter setting unit sets and/or updates the position characteristic correction parameter before correction by the correction unit is performed.

10. The biological information acquisition device according to claim 8, wherein the position characteristic correction parameter setting unit sets and/or updates the position characteristic correction parameter on a basis of a relationship between a position change amount in a case where the position of the blood flow velocity measurement unit is changed to various different positions from a reference position and a blood flow velocity measured at each of the various different positions.

11. The biological information acquisition device according to claim 7, wherein the biological information acquisition device further comprises a velocity characteristic correction parameter setting unit that sets and/or updates the velocity characteristic correction parameter.

12. The biological information acquisition device according to claim 11, wherein the velocity characteristic correction parameter setting unit sets and/or updates the velocity characteristic correction parameter before correction by the correction unit is performed.

13. The biological information acquisition device according to claim 11, wherein the velocity characteristic correction parameter setting unit sets and/or updates the velocity characteristic correction parameter on a basis of a relationship between various moving velocities of the blood flow velocity measurement unit and a blood flow velocity measured at each of the various moving velocities.

14. The biological information acquisition device according to claim 7, wherein the biological information acquisition device further comprises a transient characteristic correction parameter setting unit that sets and/or updates the transient characteristic correction parameter.

15. The biological information acquisition device according to claim 14, wherein the transient characteristic correction parameter setting unit sets and/or updates the transient characteristic correction parameter before correction by the correction unit is performed.

16. The biological information acquisition device according to claim 14, wherein the transient characteristic correction parameter setting unit sets and/or updates the transient characteristic correction parameter by fitting a blood flow velocity estimated value estimated by using the position characteristic correction parameter to temporal blood flow velocity data temporally measured in a process of changing the position of the blood flow velocity measurement unit to another position from a reference position.

17. The biological information acquisition device according to claim 1, wherein the correction unit uses the correction information to cancel a fluctuation in blood flow velocity caused by a change in position of the head and/or a fluctuation in blood flow velocity caused by a moving velocity of the head.

18. A head-mounted information presentation device comprising:
a blood flow velocity measurement unit that is installed on a head;
a detection unit that detects a position change amount of the blood flow velocity measurement unit, a moving velocity of the blood flow velocity measurement unit, or the position change amount and the moving velocity of the blood flow velocity measurement unit;
a correction information generation unit that generates correction information on a basis of the position change amount, the moving velocity, or the change amount and the moving velocity; and
a correction unit that corrects the blood flow velocity measured by the blood flow velocity measurement unit using the correction information.

19. A biological information acquisition method comprising:
a blood flow velocity measurement process of measuring a blood flow velocity of a head using a blood flow velocity measurement device installed on the head;
a detection process of detecting a position change amount of the blood flow velocity measurement device, a moving velocity of the blood flow velocity measurement device, or the position change amount and the moving velocity of the blood flow velocity measurement device;
a correction information generation process of generating correction information on a basis of the position change amount, the moving velocity, or the change amount and the moving velocity; and
a correction process of correcting the blood flow velocity measured by the blood flow velocity measurement device using the correction information.
